(19) United States
(12) Patent Application Publication
(10) Pub. No.: US 2026/0001882 A1
(43) Pub. Date: Jan. 1, 2026

(54) PROCESS FOR PREPARING BICYCLIC GLYCINE-PROLINE COMPOUNDS AND MONOCYCLIC GLYCINE-PROLINE INTERMEDIATES THEREOF

(71) Applicant: Neuren Pharmaceuticals Limited, Auckland (NZ)

(72) Inventors: Jeremy Peter SCOTT, Hoddesdon (GB); Stephen Philip KEEN, Hoddesdon (GB); George Max ESPENSEN, Hoddesdon (GB); David PARRY-JONES, Hoddesdon (GB); Ronnie Maxwell LAWRENCE, Upper Gravenhurst (GB); Clive John BLOWER, Doncaster East (AU)

(21) Appl. No.: 18/703,572

(22) PCT Filed: Oct. 21, 2022
(86) PCT No.: PCT/AU2022/051264

§ 371 Date: Apr. 22, 2024

(30) Foreign Application Priority Data
Oct. 22, 2021 (AU) 2021903383

(51) Int. Cl. C07D 487/04; C07D 207/16; C07D 487/10

(52) U.S. Cl. CPC C07D 487/04; C07D 207/16; C07D 487/10

(57) ABSTRACT

The present disclosure generally relates to a process for the synthesis of bicyclic glycine-proline compounds, and monocyclic glycine-proline intermediates thereof. In particular, the present disclosure also relates to a process for the synthesis of cyclic G-2-AllylP and its analogues. The present disclosure also relates to a process for the synthesis of optionally protected monocyclic glycine-proline intermediates. The present disclosure also relates to a process for the synthesis of esterified glycine-proline intermediates. The present disclosure also relates to compounds prepared by the processes and the use of such compounds to prepare compositions and to treat disorders.

### FIELD

The present disclosure generally relates to a process for the synthesis of bicyclic glycine-proline compounds, and monocyclic glycine-proline intermediates thereof. In particular, the present disclosure also relates to a process for the synthesis of cyclic G-2-AllylP and its analogues. The present disclosure also relates to a process for the synthesis of optionally protected monocyclic glycine-proline intermediates. The present disclosure also relates to a process for the synthesis of esterified glycine-proline intermediates. The present disclosure also relates to compounds prepared by the processes and the use of such compounds to prepare compositions and to treat disorders.

### BACKGROUND

Cyclic G-2-AllylP, also known as cG-2-AllylP and cyclo-glycyl-L-2-allylproline, is a synthetic analogue of the neurotrophic peptide, cyclic glycine proline (cGP). cGP occurs naturally in the brain and is believed to be associated with a series of neurophysiological effects, although its precise mechanism of action remains unknown.

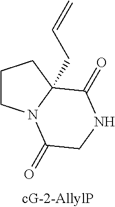

cG-2-AllylP has been investigated as the clinical candidate, NNZ-2591, and has demonstrated efficacy in pre-clinical models of Parkinson's disease, stroke, traumatic brain injury, peripheral neuropathy, Fragile X Syndrome, memory impairment, multiple sclerosis, Phelan-McDermid Syndrome, Pitt Hopkins Syndrome, Angelman Syndrome and Prader-Willi Syndrome.

To date, the reported process for preparing NNZ-2591 (cG-2-AllylP) comes from the international PCT publication WO2005/023815, and is shown in Scheme 1, below. According to the reported process, oxazolidinone 2 can be synthesised by reacting chloral with (S)-proline. This oxazolidinone 2 is then deprotonated using lithium diisopropylamide (LDA), followed by reaction with allyl bromide to produce allyl-oxazolidinone 3. Reaction of the allyl-oxazolidinone 3 with anhydrous hydrogen chloride (generated by reaction of acetyl chloride with methanol) forms the ring-opened monocyclic ester 4 as the hydrochloride salt, which is then reacted with N-boc-protected glycine 5 to form the dipeptide 6. An N-terminus deprotection of dipeptide 6 is provided using an acid, followed by a cyclisation reaction using a weak base for elimination of the methoxy group of the methyl ester, to obtain the bicyclic glycine-proline compound 1, cG-2-AllylP.

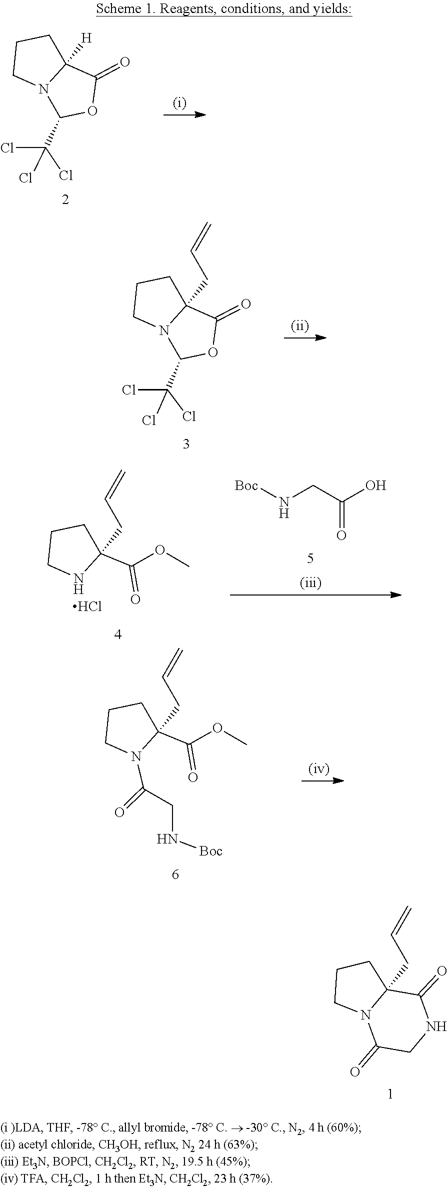

A drawback to the reported process is the inability to efficiently scale this chemistry to the quantities that are required for clinical investigation and manufacture for clinical development.

This is highlighted in particular by the yields of each reaction step, which combine to provide a total yield over the four reaction steps of approximately 6%. This is, in part, due to purification being required at each step, typically in the form of flash chromatography. Such flash chromatography is not viable in the purification of large-scale quantities. It was also found that the use of TFA (trifluoroacetic acid) and Et₃N (triethylamine) for the cyclisation step can result in a mixture of salts being formed from which it is difficult to isolate cG-2-AllylP as a free compound.

Accordingly, there remains a need for an efficient and safe scalable process for preparing cG-2-AllylP and its analogues in good yields and high purity.

### SUMMARY

The subject matter of the present disclosure is predicated in part on the development of an efficient and safe scalable process for preparing bicyclic glycine-proline compounds, including cG-2-AllylP.

It will be appreciated that other aspects, embodiments, and examples of the compounds, pharmaceutical compositions, methods, or uses, are further described herein.

In one aspect, there is provided a process for preparing a bicyclic glycine-proline compound of Formula 1 comprising a base initiated cyclisation reaction of a compound of Formula 2 to form the compound of Formula 1:

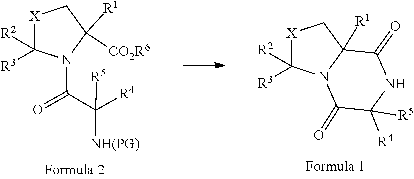

wherein X is selected from CR⁷R⁸, NR⁷, O, and S; R⁷ and R⁸ are each independently selected from hydrogen and alkyl; R¹, R², R³, R⁴, and R⁵, are each independently selected from hydrogen, halogen, alkyl, alkenyl, alkynyl, haloalkyl, —O-haloalkyl, 3-10-membered carbocycle, 3-10-membered heterocycle, —OR⁹, —SR⁹, —NR⁹R¹⁰, —NO₂, —CN, —C(O)R⁹, —C(O)OR⁹, —C(O)NR⁹R¹⁰, and —CNR⁹, or R⁴ and R⁵ taken together is a 3-10-membered carbocycle; R⁹ and R¹⁰ are each independently selected from hydrogen, —C₁₋₈alkyl, and —C₂₋₈ alkenyl; R⁶ is selected from alkyl, aryl and alkylaryl; and PG is an amine protecting group.

In a further aspect, there is provided a process for preparing an amide compound of Formula 2:

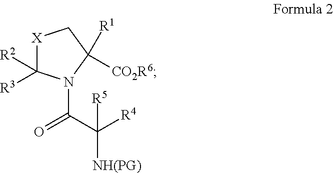

comprising reacting a compound of Formula 4 or salt thereof:

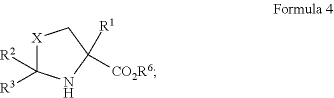

with a compound of Formula 5:

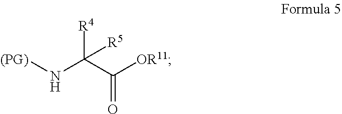

under amide coupling conditions;
wherein X is selected from CR⁷R⁸, NR⁷, O, and S; R⁷ and R⁸ are each independently selected from hydrogen and alkyl; R¹, R², R³, R⁴, and R⁵, are each independently selected from hydrogen, halogen, alkyl, alkenyl, alkynyl, haloalkyl, —O-haloalkyl, 3-10-membered carbocycle, 3-10-membered heterocycle, —OR⁹, —SR⁹, —NR⁹R¹⁰, —NO₂, —CN, —C(O)R⁹, —C(O)OR⁹, —C(O)NR⁹R¹⁰, and —CNR⁹, or R⁴ and R⁵ taken together is a 3-10-membered carbocycle; R⁹ and R¹⁰ are each independently selected from hydrogen, —C₁₋₈alkyl, and —C₂₋₈-alkenyl; R⁶ is selected from alkyl, aryl and alkylaryl; PG is an amine protecting group; and R¹¹ is selected from hydrogen and an activating group (AG).

In a further aspect, there is provided a process for preparing a compound of Formula 4 or salt thereof:

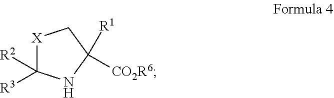

comprising reacting a compound of Formula 6 or salt thereof:

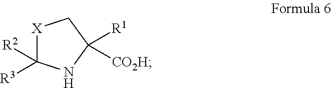

under acid-catalysed esterification conditions;
wherein X is selected from CR⁷R⁸, NR⁷, O, and S; R⁷ and R⁸ are each independently selected from hydrogen and alkyl; R¹, R², and R³, are each independently selected from hydrogen, halogen, alkyl, alkenyl, alkynyl, haloalkyl, —O-haloalkyl, 3-10-membered carbocycle, 3-10-membered heterocycle, —OR⁹, —SR⁹, —NR⁹R¹⁰, —NO₂, —CN, —C(O)R⁹, —C(O)OR⁹, —C(O)NR⁹R¹⁰, and —CNR⁹; R⁹ and R¹⁰ are each independently selected from hydrogen, —C₁₋₈alkyl, and —C₂₋₈alkenyl; and R⁶ is selected from alkyl, aryl and alkylaryl.

In other embodiments or examples, the process of preparing a compound of Formula 1 comprises the process of one or more of the above further aspects.

In another aspect, there is provided a bicyclic glycine-proline compound of Formula 1:

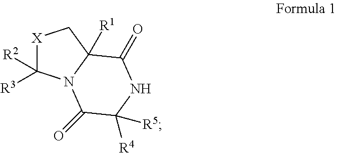

prepared by the process as described herein.

In other aspects or embodiments, there is provided a compound of Formula 2, Formula 4, Formula 5, or Formula 6, prepared from any one or more processes as described by the above aspects, or any embodiments or examples thereof as described herein.

The present invention is not to be limited in scope by the specific embodiments described herein, which are intended for the purpose of exemplification only. Functionally-equivalent products, compositions and methods are clearly within the scope of the invention as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### DETAILED DESCRIPTION

### General Definitions

Unless specifically defined otherwise, all technical and scientific terms used herein shall be taken to have the same meaning as commonly understood by one of ordinary skill in the art (e.g., chemistry, biochemistry, medicinal chemistry, microbiology and the like).

As used herein, the term “and/or”, e.g., “X and/or Y”, shall be understood to mean either “X and Y” or “X or Y” and shall be taken to provide explicit support for both meanings or for either meaning, e.g., A and/or B includes the options i) A, ii) B, or iii) A and B.

As used herein, the term “about”, unless stated to the contrary, refers to +/−20%, typically +/−10%, typically +/−5%, of the designated value.

As used herein, the terms “a”, “an”, and “the” include both singular and plural aspects, unless the context clearly indicates otherwise.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout the present specification, various aspects and components of the invention can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 5, 5.5 and 6, unless where integers are required or implicit from context. This applies regardless of the breadth of the disclosed range. Where specific values are required, these will be indicated in the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It will be clearly understood that, although a number of prior art publications are referred to herein, this reference does not constitute an admission that any of these documents form part of the common general knowledge in the art in the United States, Australia, or in any other country.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will prevail. In addition, the materials, methods, and examples are illustrative only and not intended to be limiting.

Salts may be formed in the case of embodiments of the compounds of Formulae 1 to 9, which contain a suitable acidic or basic group. Suitable salts of the compounds of Formulae 1 to 9 include those formed with organic or inorganic acids or bases. Accordingly, it will be appreciated, that in referring to a compound by its formula (i.e., Formula 1), reference is made to both the free-base/free-acid compound, and the corresponding salt thereof.

As used herein, the phrase “pharmaceutically acceptable salt” or a like term refers to pharmaceutically acceptable organic or inorganic salts. It will be appreciated that any reference to “salt” herein can include “pharmaceutically acceptable salts”. Exemplary acid addition salts include, but are not limited to, sulfate, citrate, acetate, oxalate, chloride, bromide, iodide, nitrate, bisulfate, phosphate, isonicotinate, lactate, salicylate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. Exemplary base addition salts include, but are not limited to, ammonium salts, alkali metal salts, for example those of potassium and sodium, alkaline earth metal salts, for example those of calcium and magnesium, and salts with organic bases, for example dicyclohexylamine, N-methyl-D-glucamine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterions. It will also be appreciated that non-pharmaceutically acceptable salts also fall within the scope of the present disclosure since these may be useful as intermediates in the preparation of pharmaceutically acceptable salts or may be useful during storage or transport.

Those skilled in the art of organic and/or medicinal chemistry will appreciate that many organic compounds can form complexes with solvents in which they are reacted or from which they are precipitated or crystallized. These complexes are known as “solvates”. For example, a complex with water is known as a “hydrate”. As used herein, the phrase “pharmaceutically acceptable solvate” or “solvate” refer to an association of one or more solvent molecules and a compound of the present disclosure. Examples of solvents that form pharmaceutically acceptable solvates include, but are not limited to, water, isopropyl alcohol, ethanol, methanol, dimethylsulfoxide, ethyl acetate, isopropyl acetate, acetic acid, and ethanolamine. It will be understood that the present disclosure encompasses solvated forms, including hydrates, of the compounds of Formula 1 and salts thereof.

The compounds of the present disclosure may contain chiral (asymmetric) centers or the molecule as a whole may be chiral. The individual stereoisomers (enantiomers and diastereoisomers) and mixtures of these are within the scope of the present disclosure.

As used herein, the term “stereoisomer” refers to compounds having the same molecular formula and sequence of bonded atoms (i.e., atom connectivity), though differ in the three-dimensional orientations of their atoms in space. As used herein, the term “enantiomers” refers to two compounds that are stereoisomers in that they are non-superimposable mirror images of one another. Relevant stereocenters may be denoted with (R)- or (S)-configuration.

Those skilled in the art of organic and/or medicinal chemistry will appreciate that the compounds of Formula 1 and salts thereof may be present in an amorphous form or in a crystalline form. It will be understood that the present disclosure encompasses all forms and polymorphs of the compounds of Formula 1 and salts thereof.

As used herein, the term “protecting group” has the meaning conventionally associated with it in organic synthesis/medicinal chemistry, i.e., a chemical group that selectively blocks one or more reactive sites in a multifunctional compound such that a chemical reaction can be carried out selectively on another unprotected reactive site and such that the group can readily be removed after the selective reaction is complete.

Implicit hydrogen atoms (such as the hydrogen atoms present on the pyrrole ring, etc.) are omitted from the formulae for clarity, but would be understood by the skilled person to be present.

As used herein, the term “halogen” means fluorine, chlorine, bromine, or iodine.

As used herein, the term “alkyl” encompasses both straight-chain (i.e., linear) and branched-chain hydrocarbon groups. Examples of alkyl groups include, but are not limited to, methyl, ethyl, n-propyl, iso-propyl, n-butyl, t-butyl, i-butyl, sec-butyl, pentyl, and hexyl groups. In one example, the alkyl group is of one to eight carbon atoms (i.e. C₁₋₈alkyl). In one example, the alkyl group is of one to six carbon atoms (i.e. C₁₋₆alkyl).

As used herein, the term “alkenyl” refers to both straight and branched chain unsaturated hydrocarbon groups with at least one carbon-carbon double bond. Examples of alkenyl groups include, but are not limited to, ethenyl, propenyl, butenyl, pentenyl, and hexenyl groups. In one example, the alkenyl group is of two to eight carbon atoms (i.e. C₂₋₈alkenyl). In one example, the alkenyl group is of two to six carbon atoms (i.e. C₂₋₆alkenyl).

As used herein, the term “alkynyl” refers to both straight and branched chain unsaturated hydrocarbon groups with at least one carbon-carbon triple bond. Examples of alkynyl groups include, but are not limited to, ethynyl, propynyl, butynyl, pentynyl, and hexynyl groups. In one example, the alkynyl group is of two to eight carbon atoms (i.e. C₂₋₈alkynyl). In one example, the alkynyl group is of two to six carbon atoms (i.e. C₂₋₆alkynyl).

As used herein, the term “aryl” refers to a functional group derived from an aromatic ring compound where one hydrogen atom is removed from the ring. Examples of aryl groups include, but are not limited to, phenyl, tolyl, xylyl, and naphthyl groups. In one example, the aryl group is of five to twelve carbon atoms (i.e. C₅₋₁₂aryl). In one example, the aryl group is of five to eight carbon atoms (i.e. C₅₋₈aryl).

As used herein, the term “alkylaryl” refers to a functional group derived from an alkyl-substituted aromatic ring compound where one hydrogen atom is removed from the alkyl group. Examples of alkylaryl groups include, but are not limited to, methylphenyl (benzyl), ethylphenyl, methylnaphthyl, and ethylnaphthyl groups. In one example, the alkylaryl group is of one six to fourteen carbon atoms (i.e. C₆₋₁₄alkylaryl). In one example, the alkyl group is of six to ten carbon atoms (i.e. C₆₋₁₀alkylaryl).

As used herein, the term “carbocyclyl” refers to an aromatic or non-aromatic cyclic group of carbon atoms. A carbocyclyl group may, for example, be monocyclic or polycyclic (e.g. bicyclic, tricyclic). A polycyclic carbocyclyl group may contain fused rings. In one example, the carbocyclyl group is of three to ten carbon atoms (i.e. C₃₋₁₀carbocyclyl). In one example, the carbocyclyl group is of three to seven carbon atoms (i.e. C₃₋₇carbocyclyl). Examples of monocyclic non-aromatic carbocyclyl groups include cyclopropyl, cyclobutyl, cyclopentyl, cyclopentenyl, cyclohexyl, cyclohexenyl, cycloheptyl, and cyclooctyl groups. Aromatic carbocyclyl groups include phenyl and napthalenyl.

As used herein, the term “heterocyclyl” refers to an aromatic or non-aromatic cyclic group which is analogous to a carbocyclic group, but in which from one to three of the carbon atoms is/are replaced by one or more heteroatoms independently selected from nitrogen, oxygen, or sulfur. A heterocyclyl group may, for example, be monocyclic or polycyclic (e.g. bicyclic, tricyclic). A polycyclic heterocyclyl may for example contain fused rings. In a bicyclic heterocyclyl group there may be one or more heteroatoms in each ring, or heteroatoms only in one of the rings. Heterocyclyl groups containing a suitable nitrogen atom include the corresponding N-oxides. In one example, the heterocyclyl group is of three to ten atoms (i.e. 3-10-membered heterocyclyl). In one example, the heterocyclyl group is of three to seven atoms (i.e. 3-7-membered heterocyclyl). Examples of monocyclic non-aromatic heterocyclyl groups include aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl and azepanyl. Examples of bicyclic heterocyclyl groups in which one of the rings is non-aromatic include dihydrobenzofuranyl, indanyl, indolinyl, isoindolinyl, tetrahydroisoquinolinyl, tetrahydroquinolyl, and benzoazepanyl. Examples of monocyclic aromatic heterocyclyl groups (also referred to as monocyclic heteroaryl groups) include furanyl, thienyl, pyrrolyl, oxazolyl, thiazolyl, imidazolyl, oxadiazolyl, thiadiazolyl, pyridyl, triazolyl, triazinyl, pyridazyl, isothiazolyl, isoxazolyl, pyrazinyl, pyrazolyl, and pyrimidinyl. Examples of bicyclic aromatic heterocyclyl groups (also referred to as bicyclic heteroaryl groups) include quinoxalinyl, quinazolinyl, pyridopyrazinyl, benzoxazolyl, benzothiophenyl, benzimidazolyl, naphthyridinyl, quinolinyl, benzofuranyl, indolyl, benzothiazolyl, oxazolyl[4,5-b]pyridyl, pyridopyrimidinyl, isoquinolinyl, and benzohydroxazole.

As used herein, the term “saturated” refers to a group where all available valence bonds of the backbone atoms are attached to other atoms. Representative examples of saturated groups include, but are not limited to, butyl, cyclohexyl, piperidine, and the like.

As used herein, the term “unsaturated” refers to a group where at least one valence bond of two adjacent backbone atoms is not attached to other atoms. Representative examples include, but are not limited to, alkenes (e.g., —CH₂—CH₂CH═CH), phenyl, pyrrole, and the like.

As used herein, the term “optionally substituted” refers to a group being unsubstituted or substituted as described herein.

As used herein, the term “substituted” refers to a group having one or more hydrogens or other atoms removed from a carbon or suitable heteroatom and replaced with a further group (i.e., substituent).

As used herein, the term “unsubstituted” refers to a group that does not have any further groups attached thereto or substituted therefore.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

### Process for Preparing Cyclic G-2-AllylP

The subject matter of the present disclosure is predicated in part on the discovery of an efficient and safe scalable process for preparing bicyclic glycine-proline compounds, including cG-2-AllylP. Scheme 2, below, provides an example of an efficient and safe scalable process for preparing bicyclic glycine-proline compounds of Formula 1, including cG-2-AllylP.

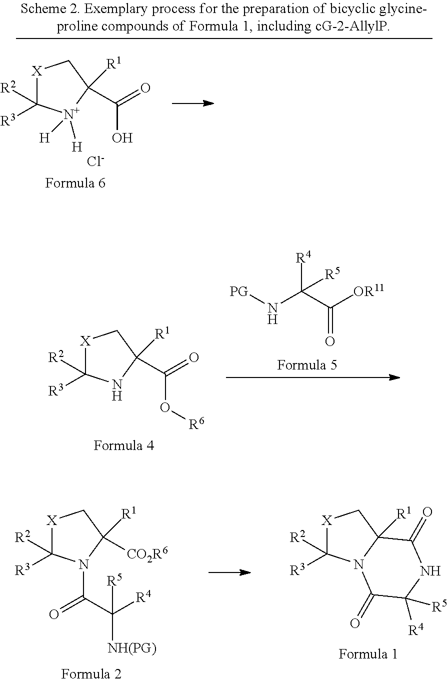

The above process is described further below in relation to each of the steps of the process. Each step may provide its own independent process aspect, embodiment, or example for preparing an intermediate or compound per se, or may provide a further embodiment or example to another process aspect or embodiment as described herein. Each intermediate or prepared compound of each step may also provide its own independent aspect, embodiment, or example, in relation to compounds, compositions, and/or processes thereof.

### Synthesis of a Compound of Formula 1

The present disclosure provides a process for preparing a bicyclic glycine-proline compound of Formula 1:

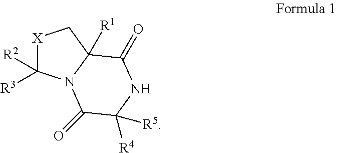

In some embodiments, the process for preparing a bicyclic glycine-proline compound of Formula 1 comprises a base initiated cyclisation reaction of a compound of Formula 2 to form the compound of Formula 1:

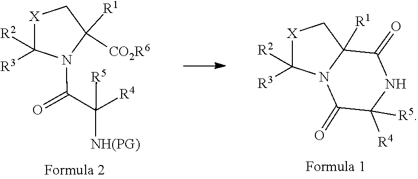

In some embodiments, the process for preparing a bicyclic glycine-proline compound of Formula 1 comprises a base initiated cyclisation reaction of a compound of Formula 2 to form the compound of Formula 1 that proceeds via an intermediate of Formula 3:

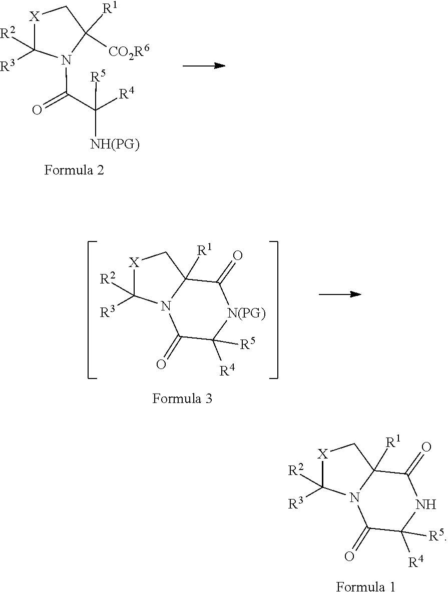

In some embodiments, the process for preparing a bicyclic glycine-proline compound of Formula 1 comprises a base initiated cyclisation reaction of a compound of Formula 2 to form a protected bicyclic glycine-proline compound of Formula 3:

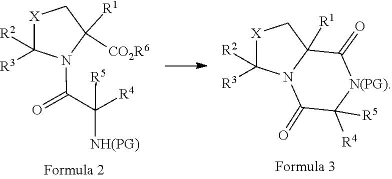

In some embodiments, the process for preparing a bicyclic glycine-proline compound of Formula 1 comprises removing the protecting group from the compound of Formula 3 to form the bicyclic glycine-proline compound of Formula 1:

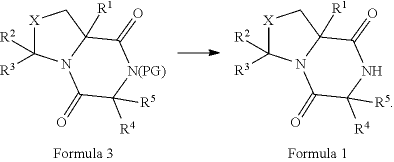

In some embodiments, X is selected from CR⁷R⁸, NR⁷, O, and S. In one example, X is CR⁷R⁸. In one example, X is NR⁷. In one example, X is O. In one example, X is S. In some embodiments, R⁷ and R⁸ are each independently selected from hydrogen and alkyl. In one example, R⁷ is hydrogen. In some embodiments, R⁷ is alkyl. In one example, R⁸ is hydrogen. In some embodiments, R⁸ is alkyl. Accordingly, in one example, X is CH₂. In one example, X is NH.

In some embodiments, R¹, R², R³, R⁴, and R⁵, are each independently selected from hydrogen, halogen, alkyl, alkenyl, alkynyl, haloalkyl, —O-haloalkyl, 3-10-membered carbocycle, 3-10-membered heterocycle, —OR⁹, —SR⁹, —NR⁹R¹⁰, —NO₂, —CN, —C(O)R⁹, —C(O)OR⁹, —C(O)NR⁹R¹⁰, and —CNR⁹, or R⁴ and R⁵ taken together is a 3-10-membered carbocycle. In some embodiments, R¹ is alkyl. In some embodiments, R¹, R², R³, R⁴, and R⁵, are each independently selected from hydrogen, halogen, alkyl, alkenyl, haloalkyl, and R⁴ and R⁵ taken together is a 3-10-membered carbocycle. In some embodiments, R¹ is hydrogen, alkyl or alkenyl. In some embodiments, R¹ is alkenyl. In one example, R¹ is —CH₂—CH=CH₂. In one example, R¹ is —CH₃. In one example, R² is hydrogen. In one example, R³ is hydrogen. In one example, R⁴ is hydrogen. In one example, R⁵ is hydrogen. In some embodiments, R² is alkyl. In some embodiments, R³ is alkyl. In some embodiments, R⁴ is alkyl. In some embodiments, R⁵ is alkyl. In one example, R⁴ and R⁵ taken together are a cyclopentyl group. In one example, R⁴ and R⁵ taken together are a cyclohexyl group.

In some embodiments, R⁹ and R¹⁰ are each independently selected from hydrogen, —C₁₋₈alkyl, and —C₂₋₈alkenyl. In one example, R⁹ is hydrogen. In some embodiments, R⁹ is —C₁₋₈alkyl. In one example, R¹⁰ is hydrogen. In some embodiments, R¹⁰ is —C₁₋₈alkyl.

In some embodiments, R⁶ is selected from alkyl, aryl and alkylaryl. In one example, R⁶ is alkyl. In one example, R⁶ is C₁₋₆alkyl. In one example, R⁶ is —CH₃. In one example, R⁶ is —CH₂CH₃. In one example, R⁶ is aryl. In one example, R⁶ is phenyl. In one example, R⁶ is alkylaryl. In one example, R⁶ is benzyl.

In one example, the compound of Formula 1 is selected from:

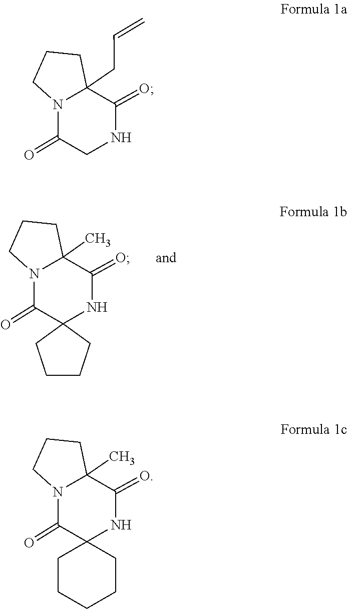 [SMILES: C=CCC12CCCN1C(=O)CNC2=O ; CC12CCCN1C(=O)C1(CCCC1)NC2=O ; CC12CCCN1C(=O)C1(CCCCC1)NC2=O]

Accordingly, in one example, X is CH₂, R¹ is —CH₂—CH═CH₂, and R², R³, R⁴, and R⁵ are each hydrogen. In one example, X is CH₂, R¹ is —CH₃, R² and R³ are hydrogen, and R⁴ and R⁵ taken together are a cyclopentyl group. In one example, X is CH₂, R¹ is —CH₃, R² and R³ are hydrogen, and R⁴ and R⁵ taken together are a cyclohexyl group.

PG is an amine protecting group. The term “amine protecting group” specifically refers to a protecting group that chemically modifies an amine functional group to obtain chemoselectivity in a subsequent chemical reaction. Examples of amine protecting groups include, but are not limited to, carbamate, amide, sulfonamide, benzyl, benzylidene, and trityl (Trt) protecting groups. In some embodiments, the protecting group (PG) is an amide, sulfonamide, trityl (Trt) or carbamate protecting group. Examples of carbamate protecting groups include, but are not limited to, 9-fluorenylmethyloxycarbonyl (Fmoc), tert-butyloxycarbonyl (Boc), allyloxycarbonyl (Alloc), benzyl carbamate (Cbz), and p-methoxybenzyl carbonyl (MeOZ) groups. Examples of sulfonamide protecting groups include para-tosyl (p-toluenesulfonyl) and mesyl groups. Examples of amide protecting groups include a trifluoroacetyl (TFA) group. In one example, the amine protecting group (PG) is a tert-butyloxycarbonyl (Boc) protecting group. In one example, the amine protecting group (PG) is an -Fmoc (fluorenylmethyloxycarbonyl) protecting group. In one example, the amine protecting group (PG) is a -Cbz (carboxybenzyl) protecting group. In one example, the amine protecting group (PG) is a para-tosyl protecting group. In one example, the amine protecting group (PG) is a benzyl group. In one example, the amine protecting group (PG) is a trityl (Trt) group. In one example, the amine protecting group (PG) is a trifluoroacetyl (TFA) group.

It will be appreciated that the base initiated cyclisation reaction is initiated by the reaction of a base directly with the compound of Formula 2. For example, the “base initiated cyclisation reaction” does not involve a stepwise approach of a first step of acid initiated removal of the PG group from the compound of Formula 2 before undertaking a subsequent cyclisation step using a weak base such as triethylamine (Et₃N). One advantage of the base initiated reaction has been to enable a cyclisation and then PG deprotection concurrently in the same reaction vessel (e.g. a one-pot single reaction system for both cyclisation and deprotection). Another advantage of the base initiated reaction has been to enable the use of reduced amounts of base for the cyclisation, which requires reduced amounts of acid during workup of the reaction mixture in neutralising and quenching the base used during the reaction. The base initiated reaction has also enabled good yields and high purities.

In some embodiments, the base initiated cyclisation reaction is performed in the presence of a strong base. A strong base may be defined by the pKa value of the conjugate acid of the base, which is defined as its pKaH. pKaH is the negative log of the acid dissociation constant (Ka), and a lower pKaH value indicates a stronger conjugate acid (i.e., the acid more fully dissociates in water), and therefore a weaker base, and a higher pKaH value indicates a weaker conjugate acid, and therefore, a stronger base. In some embodiments, the pKaH value of the base is greater than about 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39. In some embodiments, the base has a pKaH value of less than about 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10. The base may be selected from a base having a pKaH value in a range provided by any two of these upper and/or lower ranges, for example between about 9 and 40, between about 9 and 37, between about 9 and 18, between about 13 and 40, between about 13 and 28, or between about 30 and 40.

In some embodiments, the base is an anionic base. In some embodiments, the base is provided by a metal salt comprising a metal cation and counter anion selected from a carbonate, carbanion, amine, hydride, silylamide, alkoxide, hydroxide, oxide, or phenoxide. In some embodiments, the base is a compound comprising a non-metal element and a group selected from a carbonate, carbanion, amine, hydride, silylamide, alkoxide, hydroxide, oxide, or phenoxide. In some embodiments, the base is a metal salt comprising a Group 1 metal cation and counter anion selected from a carbonate, carbanion, amine, hydride, silylamide, alkoxide, hydroxide, oxide, or phenoxide. In one example, the Group 1 metal is lithium, sodium, potassium, or caesium. In one example, the Group 1 metal is sodium. In one example, the Group 1 metal is potassium. In some embodiments, the base is a metal alkoxide. The metal alkoxide may for example be selected from sodium methoxide (NaOMe), sodium ethoxide (NaOEt), sodium isopropoxide, and sodium tert-butoxide (NaOtBu). In some embodiments, the base is a silylamide. The metal silylamide may be selected from lithium bis(trimethylsilyl)amide (lithium hexamethyldisilazide or LiHMDS), sodium bis(trimethylsilyl)amide (sodium hexamethyldisilazide or NaHMDS), and potassium bis(trimethylsilyl)amide (potassium hexamethyldisilazide or KHMDS). In some embodiments, the base is a carbonate. The carbonate may for example be selected from lithium carbonate, sodium carbonate, potassium carbonate and caesium carbonate. In some embodiments, the base is a metal hydroxide. The metal hydroxide may for example be selected from sodium hydroxide and potassium hydroxide. In some embodiments, the base is a hydride. The hydride may for example be selected from sodium hydride and potassium hydride. In some embodiments, the base is an amine. The amine may for example be selected from sodamide (NaNH₂) and lithium diisopropylamide (LDA). In some embodiments, the base is a metal oxide. The metal oxide may for example be selected from sodium oxide and potassium oxide. In some embodiments, the base is a phenoxide. The phenoxide may for example be selected from sodium phenoxide and potassium phenoxide.

In some examples, the base is selected from sodium methoxide (NaOMe), sodium ethoxide (NaOEt), sodium isopropoxide, sodium tert-butoxide (NaOtBu), sodium bis(trimethylsilyl)amide (NaHMDS), lithium bis(trimethylsilyl)amide (LiHMDS), sodium hydride (NaH), sodium hydroxide (NaOH), potassium hydroxide (KOH), potassium carbonate, lithium diisopropylamide (LDA), potassium methoxide (KOMe), potassium ethoxide (KOEt), potassium isopropoxide, potassium tert-butoxide (KOtBu), and any combinations thereof. In one example, the base is sodium methoxide (NaOMe). In one example, the base is sodium ethoxide (NaOEt). In one example, the base is sodium isopropoxide. In one example the base is sodium tert-butoxide (NaOtBu). In one example, the base is sodium bis(trimethylsilyl)amide (NaHMDS). In one example, the base is lithium bis(trimethylsilyl)amide (LiHMDS). In one example, the base is sodium hydride (NaH). In one example, the base is lithium diisopropylamide (LDA). In one example, the base is potassium carbonate. In one example, the base is potassium tert-butoxide (KOtBu).

The alkoxide may be a C₁₋₂₀alkoxide, C₁₋₁₀alkoxide, C₁₋₆alkoxide, or C₁₋₄alkoxide. In one example, the alkoxide comprises a Group 1 metal, a Group 2 metal, a transition metal, a post-transition metal or a metalloid. In another example, the alkoxide comprises lithium, sodium, potassium, aluminium, molybdenum, silicon, titanium, or tungsten. In another example, the alkoxide comprises a Group 1 metal, for example lithium, sodium, or potassium. In another example, the alkoxide comprises a non-Group 1 element, such as a Group 2 metal, a transition metal, a post-transition metal or a metalloid. In another example, the alkoxide comprises a non-Group 1 element, for example aluminium, molybdenum, silicon, titanium, or tungsten. In another example, the alkoxide is selected from aluminium isopropoxide, hexa(tert-butoxy)dimolybdenum(III), hexa(tert-butoxy)ditungsten(III), lithium methoxide, lithium tert-butoxide, rubidium methoxide, caesium methoxide, sodium methoxide, sodium ethoxide, sodium tert-butoxide, sodium isopropoxide, methyltrimethoxysilane, potassium methoxide, potassium ethoxide, potassium isopropoxide, potassium tert-butoxide, silicon methoxide (tetramethyl orthosilicate), silicon ethoxide (tetraethyl orthosilicate), sodium bis(2-methoxyethoxy)aluminium hydride, sodium dehydroacetate, titanium butoxide, titanium isopropoxide (titanium tetraisopropoxide or TTIP), or any combinations thereof.

As will be appreciated by the person skilled in the art, the process will be performed in a suitable solvent. In some embodiments, the cyclisation reaction is performed in a solvent, such as a non-polar solvent, polar protic solvent, or polar aprotic solvent. In some embodiments, the cyclisation reaction is performed in a protic or aprotic solvent. The aprotic solvent may be polar or non-polar. Examples of polar protic solvents include, but are not limited to, alcohols, glycols, and any combinations thereof. Examples of alcohols include, but are not limited to, methanol (MeOH), ethanol (EtOH), 1-propanol, isopropyl alcohol (2-propanol, iPrOH or IPA), 1-butanol, 2-butanol, t-butanol (t-BuOH), 1-pentanol, 3-methyl-1-butanol, 2-methyl-1-propanol, and combinations thereof. Examples of glycols include, but are not limited to, ethylene glycol. In one example, the cyclisation reaction is performed in an alcohol. In one example, the cyclisation reaction is performed in methanol. In one example, the cyclisation reaction is performed in isopropyl alcohol. In one example, the cyclisation reaction is performed in ethanol. In some embodiments, the cyclisation reaction is performed in a polar or non-polar aprotic solvent. In some embodiments, the cyclisation reaction is performed in a non-polar ether solvent. In some embodiments, the cyclisation reaction is performed in a polar or non-polar halogenated hydrocarbon solvent (e.g. chlorocarbon solvent such as dichloromethane). In some embodiments, the cyclisation reaction is performed in a non-polar hydrocarbon solvent. In some embodiments, the cyclisation reaction is performed in a non-polar aromatic hydrocarbon solvent. In some embodiments, the cyclisation reaction is performed in any combination of polar protic, polar aprotic, non-polar ether or halogenated solvents. In some embodiments, the cyclisation reaction is performed in any combination of hydrocarbon, aromatic hydrocarbon, polar aprotic, non-polar ether, or halogenated solvents. Examples of polar aprotic solvents include, but are not limited to, halogenated hydrocarbons, ketones, nitriles, esters, carbonate esters, ethers, sulfoxides, sulfones, amides, nitroalkanes, pyrrolidines, pyridines, and combinations thereof. Examples of non-polar solvents include, but are not limited to, hydrocarbons and aromatic hydrocarbons and combinations thereof. Examples of ketones include, but are not limited to, acetone, methylethyl ketone (MEK), methylbutyl ketone (MBK), methylisobutyl ketone (MIBK), methylisopropyl ketone (MIPK), and combinations thereof. Examples of nitriles include, but are not limited to, acetonitrile (MeCN). Examples of esters include, but are not limited to, ethyl formate, methyl acetate (MeOAc), ethyl acetate (EtOAc), propyl acetate, isopropyl acetate (iPAC), n-butyl acetate, isobutyl acetate, and combinations thereof. Examples of carbonate esters include, but are not limited to, dimethyl carbonate (DMC), propylene carbonate (PC), and combinations thereof. Examples of polar and non-polar ethers include, but are not limited to, methyl-tert-butyl ether (MTBE), diethyl ether, 1,4-dioxane, 2-methoxyethanol, 2-ethoxyethanol, dimethoxyethane (DME or monoglyme), 1,1-dimethoxymethane, 2,2-dimethoxypropane, 1,1-diethoxypropane, isopropyl ether, petroleum ether, cyclopentyl methyl ether (CPME), anisole (methoxybenzene), methyltetrahydrofuran (MeTHF), tetrahydrofuran (THF), and combinations thereof. Examples of sulfoxides include, but are not limited to, dimethylsulfoxide (DMSO). Examples of sulfones include, but are not limited to, sulfolane. Examples of amides include, but are not limited to, formamide, N,N-dimethylacetamide, N,N-dimethylformamide (DMF), and combinations thereof. Examples of nitroalkanes include, but are not limited to, nitromethane. Examples of pyrrolidines include, but are not limited to, N-methylpyrrolidone (NMP). Examples of pyridines include, but are not limited to, pyridine. Examples of polar and non-polar halogenated hydrocarbons, such as chlorocarbons, include, but are not limited to, dichloromethane (DCM), chloroform, 1,2-dichloroethane, 1,1,1-trichloroethane, 1,1-dichloroethene, 1,2-dichloroethene, and combinations thereof. Examples of hydrocarbons include, but are not limited to, hexanes and n-heptane and combinations thereof. Examples of aromatic hydrocarbons include, but are not limited to, toluene, xylenes and cumene and combinations thereof. In one example, the cyclisation reaction is performed in an ether. In one example, the cyclisation reaction is performed in tetrahydrofuran (THF). In one example, the cyclisation reaction is performed in methyltetrahydrofuran (MeTHF). In one example, the cyclisation reaction is performed in methyl-tert-butyl ether (MTBE). In one example, the cyclisation reaction is performed in an alkyl nitrile. In one example, the cyclisation reaction is performed in acetonitrile (MeCN). In one example, the cyclisation reaction is performed in an aromatic hydrocarbon. In one example, the cyclisation reaction is performed in toluene. In one example, the cyclisation reaction is performed in a hydrocarbon. In one example, the cyclisation reaction is performed in hexanes. In one example, the cyclisation reaction is performed in n-heptane. In one example, the cyclisation reaction is performed in a pyridine. In one example, the cyclisation reaction is performed in pyridine. In one example, the cyclisation reaction is performed in an ester. In one example, the cyclisation reaction is performed in ethyl acetate (EtOAc). In one example, the cyclisation reaction is performed in a ketone. In one example, the cyclisation reaction is performed in methyl isopropyl ketone (MIPK). In one example, the cyclisation reaction is performed in an amide. In one example, the cyclisation reaction is performed in N,N-dimethylformamide (DMF). In one example, the cyclisation reaction is performed in a sulfoxide. In one example, the cyclisation reaction is performed in dimethylsulfoxide (DMSO). In one example, the cyclisation reaction is performed in halogenated hydrocarbon. In one example, the cyclisation reaction is performed in dichloromethane (DCM). In one example, the cyclisation reaction is performed in a pyrollidine. In one example, the cyclisation reaction is performed in N-methylpyrrolidone. In one example, the cyclisation reaction is performed in a carbonate ester. In one example, the cyclisation reaction is performed in dimethyl carbonate (DMC).

The solvent may be present in the reaction in any amount suitable so as to effect the cyclisation reaction. In some examples the solvent may be anhydrous. For example, the amount of water in the solvent may be less than about (in ppm) 100, 75, 50, 25, 10, 5, 1, 0.1, or 0.01.

In some embodiments, the solvent is present in volume equivalents (L), relative to the molar amount of the compound of Formula 2, of less than about 30, 25, 20, 15, 10, 9, 8, 7, 6, 5, 4, 3, 2, or 1. In some embodiments, the solvent is present in volume equivalents (L), relative to the molar amount of the compound of Formula 2, of more than about 1, 2, 3, 4, 5, 6, 7, 8, 9, 10, 15, 20, or 25. The solvent may be in a range provided by any two or more of the upper and/or lower amounts, for example between about 1 and 30, between about 2 and 20, or between about 5 and 20.

In some embodiments, the solvent is present in volumes (L) of greater than about 1, 10, 50, 75, 100, 250, 500, 750, 1000, 2000, 3000, 4000, or 5000. In some embodiments, the solvent is present in volumes (L) of less than about 10000, 5000, 4000, 3000, 2000, 1000, 750, 500, 250, or 100. The solvent may be in a range provided by any two or more of the upper and/or lower amounts, for example between about 1 and 10000, between about 100 and 5000, or between about 500 and 2000. These volumes may relate to a single batch reaction system. It will be appreciated that multiple batch reactions may be combined.

The base may be provided in the reaction in any amount suitable so as to effect the cyclisation reaction. In some embodiments, the base is provided in molar equivalents, relative to the molar amount of the compound of Formula 2, of less than about 5, 4, 3, 2, 1.5, 1.0, or 0.5. In some embodiments, the base is provided in molar equivalents, relative to the molar amount of the compound of Formula 2, of greater than about 0.01, 0.05, 0.1, 0.3, 0.5, 1, 1.5, 2, or 3. The base may be in a range provided by any two of these upper and/or lower ranges, for example between about 0.01 and 5, between about 0.05 and 4, or between about 0.1 and 2, or between about 0.5 to 1.5.

In some embodiments, the solvent is present in volume equivalents (L), relative to the molar amount of the base used for the base initiated cyclisation, of less than about 50, 45, 40, 35, 30, 25, 20, 15, 10, 5, 2, or 1. In some embodiments, the solvent is present in volume equivalents (L), relative to the molar amount of the base used for the base initiated cyclisation, of more than about 1, 2, 5, 10, 15, 20, 25, 30, 35, or 40. The solvent may be in a range provided by any two or more of the upper and/or lower amounts, for example between about 1 and 50, between about 2 and 30, or between about 5 and 20.

In some embodiments, the concentration of base in the reaction solution (mol/L) is greater than about 0.001, 0.005, 0.01, 0.05, 0.1, 0.2, 0.3, 0.4, 0.5, 0.6, 0.7, 0.8, 0.9, 1, 2, 3, or 4. In some embodiments, the concentration of base in the reaction solution (mol/L) is less than about 5, 4, 3, 2, 1, 0.9, 0.8, 0.7, 0.6, 0.5, 0.4, 0.3, 0.2, 0.1, 0.05, or 0.01. The base may be in a concentration range provided by any two of these upper and/or lower values, for example between about 0.001 and 5, between about 0.005 and 2, or between about 0.01 and 1.

In some embodiments, the concentration of the compound of Formula 2 in the reaction solution (mol/L) is greater than about 0.001, 0.005, 0.01, 0.05, 0.1, 0.2, 0.3, 0.4, 0.5, 0.6, 0.7, 0.8, 0.9, 1, 2, 3, or 4. In some embodiments, the concentration of the compound of Formula 2 in the reaction solution (mol/L) is less than about 5, 4, 3, 2, 1, 0.9, 0.8, 0.7, 0.6, 0.5, 0.4, 0.3, 0.2, 0.1, 0.05, or 0.01. The base may be in a concentration range provided by any two of these upper and/or lower values, for example between about 0.001 and 5, between about 0.005 and 2, or between about 0.01 and 1.

The person skilled in the art will appreciate that it may be necessary to apply heat to facilitate the reaction. The amount of heat required may depend upon the solvent in which the reaction is performed in, as discussed above. In some embodiments, the cyclisation reaction is heated to between about 30° C. and 100° C., about 40° C. and 80° C., about 50° C. and 70° C., or about 55° C. and 65° C. In one example, the cyclisation reaction is heated to between about 55° C. and 65° C. In one example, the cyclisation reaction is performed in methanol and heated to between about 55° C. and 65° C.

It will be appreciated that in addition to the cyclisation, the base initiated reaction also removes the protecting group from the compound of Formula 3 to form the bicyclic glycine-proline compound of Formula 1.

In one example, the removal or deprotection of the protecting group (PG) is performed in the presence of a base. In one example, the deprotection of the protecting group (PG) is performed in the presence of the base utilised for the cyclisation reaction. That is, in such an instance, the cyclisation reaction and deprotection of the protecting group (PG) is performed in the same reaction media (i.e., in situ) with the same solvents and reagents. In such an instance, it will be appreciated that the cyclisation compound of Formula 3 does not need to be isolated prior to the deprotection of the protecting group (PG). That is, the compound of protected bicyclic glycine-proline compound of Formula 3 can be carried through in the same one-pot reaction to afford the bicyclic glycine-proline compound of Formula 1. For example, the cyclisation and deprotection can be conducted as a single reaction step in the presence of the same reagent.

The base may be neutralised with an acid once the reaction has completed. For example, when the cyclisation and deprotection is performed with sodium methoxide, the remaining sodium methoxide can be neutralised or quenched with an acid. In some embodiments, the reaction mixture is cooled to room temperature, and acid is added to the reaction mixture. In one example, the acid is hydrogen chloride in isopropyl alcohol. In one example, the acid is 6 N hydrogen chloride in isopropyl alcohol. The resultant salt by-product (e.g., sodium chloride) may be removed via filtration, to afford the bicyclic glycine-proline compound of Formula 1.

The process for preparing a bicyclic glycine-proline compound of Formula 1, as described above, involves an initial cyclisation reaction of Formula 2. This can be achieved by an initial cyclisation reaction via deprotonation of the carbamate nitrogen in the protected dipeptide. That is, the protected amine group can react with the ester group so as to form a protected cyclic amide, to obtain a compound of Formula 3. The cyclised compound of Formula 3 can retain the amine protecting group, which can be removed in-situ to form the bicyclic glycine-proline compound of Formula 1. This sequence of cyclisation and subsequent deprotection of the protecting group can provide advantages such as improved yield and purity of the bicyclic glycine-proline compound of Formula 1. It will be appreciated that other reagents may be used to facilitate deprotection in addition to the base.

In some examples, the compound of Formula 3 may be isolated prior to a PG group deprotection. In some examples, the compound of Formula 3 may be reacted in a subsequent reaction in situ or in crude form. In some examples, the compound of Formula 3 may be reacted without isolation and/or purification (i.e., reacted in situ).

Following the step of acid quenching and neutralisation of the base initiated cyclisation reaction to form the bicyclic compound of Formula 1, the compound of Formula 1 may be isolated according to any suitable means as would be understood by the person skilled in the art. For example, isolation of the compound of Formula 1 may be achieved by chromatography (e.g. reverse-phase or normal phase column chromatography), extraction, or recrystallization. In one example, the step of isolating the compound of Formula 1 is achieved by recrystallization. In recrystallising the compound of Formula 1, the person skilled in the art would appreciate that a suitable solvent includes that in which the compound of Formula 1 has a reduced solubility. In one example, the step of isolating the compound of Formula 1 is achieved by recrystallization. In some embodiments, the step of isolating the compound of Formula 1 is achieved by recrystallization wherein the solvent is selected from n-heptane, ethanol, isopropyl alcohol, toluene, methyl-t-butyl ether, acetone, methylethyl ketone, isopropyl acetate, and combinations thereof. In one example, the step of isolating the compound of Formula 1 is achieved by recrystallization wherein the solvent is a combination of isopropyl alcohol and n-heptane. In one example, the step of isolating the compound of Formula 1 is achieved by recrystallization wherein the solvent is a combination of ethanol and n-heptane. In one example, the step of isolating the compound of Formula 1 is achieved by recrystallization wherein the solvent is a combination of acetone and n-heptane. In one example, the step of isolating the compound of Formula 1 is achieved by recrystallization wherein the solvent is a combination of methylethyl ketone and n-heptane. In one example, the step of isolating the compound of Formula 1 is achieved by recrystallization wherein the solvent is isopropyl acetate.

In one example, the bicyclic glycine-proline compound of Formula 1 is a bicyclic glycine-proline compound of Formula 1a:

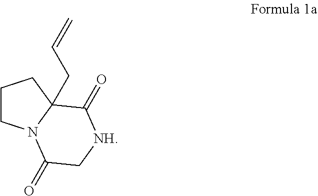

The process can comprise a cyclisation reaction of a compound of Formula 2a to form a protected bicyclic glycine-proline compound of Formula 3a:

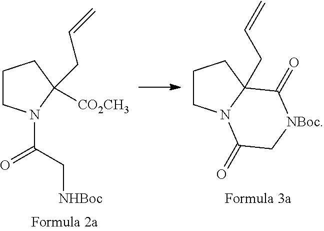 [SMILES: C=CCC1(C(=O)OC)CCCN1C(=O)CNC(=O)OC(C)(C)C ; C=CCC12CCCN1C(=O)CN(C(=O)OC(C)(C)C)C2=O]

The process can further comprise removing the protecting group from the compound of Formula 3a to form the bicyclic glycine-proline compound of Formula 1a:

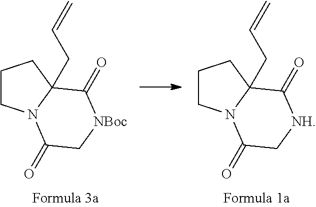 [SMILES: C=CCC12CCCN1C(=O)CN(C(=O)OC(C)(C)C)C2=O ; C=CCC12CCCN1C(=O)CNC2=O]

In one example, the bicyclic glycine-proline compound of Formula 1 is a bicyclic glycine-proline compound of Formula 1a(R) (i.e., cG-2-AllylP):

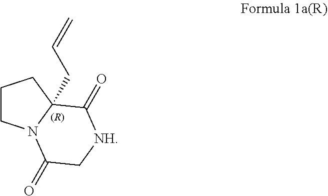

The process can comprise a cyclisation reaction of a compound of Formula 2a(R) to form a protected bicyclic glycine-proline compound of Formula 3a(R):

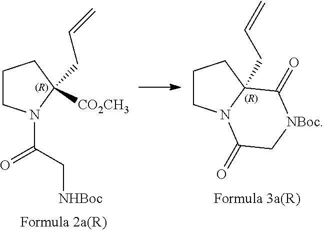

The process can further comprise removing the protecting group from the compound of Formula 3a(R) to form the bicyclic glycine-proline compound of Formula 1a(R):

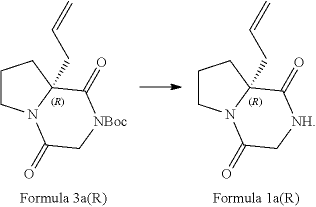

In one example, the bicyclic glycine-proline compound of Formula 1 is a bicyclic glycine-proline compound of Formula 1a(S):

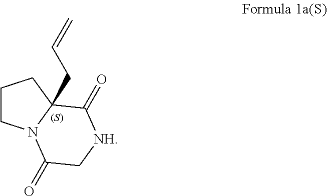

The process can comprise a cyclisation reaction of a compound of Formula 2a(S) to form a protected bicyclic glycine-proline compound of Formula 3a(S):

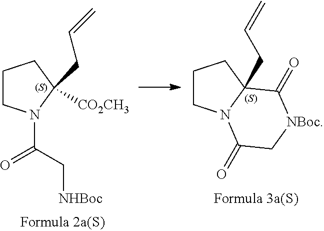

The process can further comprise removing the protecting group from the compound of Formula 3a(S) to form the bicyclic glycine-proline compound of Formula 1a(S):

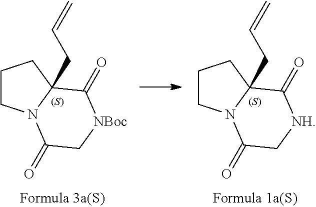

### Synthesis of a Compound of Formula 2

In some embodiments, the process for preparing an amide compound of Formula 2:

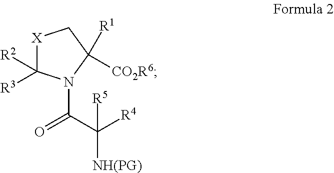

comprises reacting a compound of Formula 4 or salt thereof:

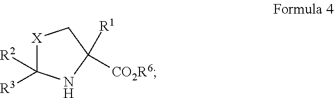

with a compound of Formula 5:

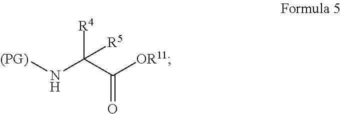

under amide coupling conditions.

In some embodiments, X is selected from CR⁷R⁸, NR⁷, O, and S. In one example, X is CR⁷R⁸. In one example, X is NR⁷. In one example, X is O. In one example, X is S. In some embodiments, R⁷ and R⁸ are each independently selected from hydrogen and alkyl. In one example, R⁷ is hydrogen. In some embodiments, R⁷ is alkyl. In one example, R⁸ is hydrogen. In some embodiments, R⁸ is alkyl. Accordingly, in one example, X is CH₂. In one example, X is NH.

In some embodiments, R¹, R², R³, R⁴, and R⁵, are each independently selected from hydrogen, halogen, alkyl, alkenyl, alkynyl, haloalkyl, —O-haloalkyl, 3-10-membered carbocycle, 3-10-membered heterocycle, —OR⁹, —SR⁹, —NR⁹R¹⁰, —NO₂, —CN, —C(O)R⁹, —C(O)OR⁹, —C(O)NR⁹R¹⁰, and —CNR⁹, or R⁴ and R⁵ taken together is a 3-10-membered carbocycle. In some embodiments, R¹ is alkyl. In some embodiments, R¹, R², R³, R⁴, and R⁵, are each independently selected from hydrogen, halogen, alkyl, alkenyl, haloalkyl, and R⁴ and R⁵ taken together is a 3-10-membered carbocycle. In some embodiments, R¹ is hydrogen, alkyl or alkenyl. In some embodiments, R¹ is alkenyl. In one example, R¹ is —CH₂—CH=CH₂. In one example, R¹ is —CH₃. In one example, R² is hydrogen. In one example, R³ is hydrogen. In one example, R⁴ is hydrogen. In one example, R⁵ is hydrogen. In some embodiments, R² is alkyl. In some embodiments, R³ is alkyl. In some embodiments, R⁴ is alkyl. In some embodiments, R⁵ is alkyl. In one example, R⁴ and R⁵ taken together are a cyclopentyl group. In one example, R⁴ and R⁵ taken together are a cyclohexyl group.

In some embodiments, R⁹ and R¹⁰ are each independently selected from hydrogen, —C₁₋₈alkyl, and —C₂₋₈alkenyl. In one example, R⁹ is hydrogen. In some embodiments, R⁹ is —C₁₋₈alkyl. In one example, R¹⁰ is hydrogen. In some embodiments, R¹⁰ is —C₁₋₈alkyl.

In some embodiments, R⁶ is selected from alkyl, aryl and alkylaryl. In one example, R⁶ is alkyl. In one example, R⁶ is C₁₋₆alkyl. In one example, R⁶ is —CH₃. In one example, R⁶ is —CH₂CH₃. In one example, R⁶ is aryl. In one example, R⁶ is phenyl. In one example, R⁶ is alkylaryl. In one example, R⁶ is benzyl.

In some embodiments, R¹¹ is selected from hydrogen and AG. In one example, R¹¹ is hydrogen. In one example, R¹¹ is AG.

As used herein, the term AG refers to an activating group. As used herein, the term “activating group” or “AG” specifically refers to a group that chemically modifies a carboxylic acid functional group to form an activated ester, which may, for example, enhance reactivity toward amines to give amides. Examples of activating groups include, but are not limited to, nitrophenyl and imide activating groups. In some embodiments, the activating group (AG) is a nitrophenyl or imide activating group. In some embodiments, the activating group (AG) is a p-nitrophenyl (para-nitrophenyl) or succinimide activating group. In one example, the activating group (AG) is a p-nitrophenyl activating group, wherein R¹¹ is a p-nitrophenyl group. In one example, the activating group (AG) is a succinimide activating group, wherein R¹¹ is a succinimide group.

In some embodiments, the process for preparing an amide compound of Formula 2 comprises reacting an activated ester compound of Formula 5 with a compound of Formula 4. In one example, the activated ester compound of Formula 5 is a p-nitrophenyl ester, wherein R¹¹ is a p-nitrophenyl group. In one example, the activated ester compound of Formula 5 is a succinimide ester, wherein R¹¹ is a succinimide group. In one example, the activated ester compound of Formula 5 is a p-nitrophenyl ester, wherein R¹¹ is a p-nitrophenyl group, and the amine protecting group PG is a butyloxycarbonyl (Boc) group. In one example, the activated ester compound of Formula 5 is a succinimide ester, wherein R¹¹ is a succinimide group, and the amine protecting group PG is a butyloxycarbonyl (Boc) group. In one example, the activated ester compound of Formula 5 is a succinimide ester, wherein R¹¹ is a succinimide group, and the amine protecting group PG is a para-tosyl (p-toluenesulfonyl; Tos) group. In one example, the activated ester compound of Formula 5 is a succinimide ester, wherein R¹¹ is a succinimide group, and the amine protecting group PG is a carboxybenzyl (Cbz) group.

PG, R⁴, or R⁵, for a compound of Formula 5 may be defined according to any examples defined elsewhere herein. In some examples, R⁴ and R⁵ are selected from hydrogen and alkyl, or together form a cycloalkyl group, such as a cyclopentyl or cyclohexyl group. In some examples, Formula 5 is compound Formula 5b wherein PG is a BOC group and wherein R¹¹ is a hydrogen:

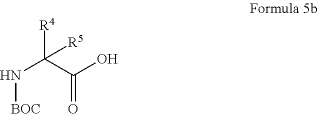

In some embodiments, the process for preparing an amide compound of Formula 2 further comprises a coupling reagent. As used herein, the term “coupling reagent” refers to a compound that promotes a chemical bond between two chemical moieties. In one example, the coupling reagent is an “amide coupling reagent” and provides a chemical bond between a carboxylic acid moiety and an amine moiety, thereby forming an amide bond. In some embodiments, the coupling reagent is a carbodiimide, or salt thereof. In some embodiments, the carbodiimide coupling reagent is selected from dicyclohexylcarbodiimide (DCC), diisopropylcarbodiimide (DIC), and 1-ethyl-3-(3-dimethylaminopropyl) carbodiimide (EDC), or salt thereof. In some embodiments, the coupling reagent is an anhydride. In some embodiments, the anhydride coupling reagent is selected from propyl phosphonic anhydride (T₃P). In some embodiments, the coupling reagent is a phosphonium. In some embodiments, the phosphonium coupling reagent is selected from benzotriazol-1-yloxy)tris(dimethylamino)phosphonium hexafluorophosphate (BOP), 1H-benzotriazol-1-yloxytripyrrolidinyl hexafluorophosphate (PyBOP), 7-Azabenzotriazol-1-yloxy)tripyrrolidinophosphonium hexafluorophosphate (PyAOP), bromotripyrrolidinophosphonium hexafluorophosphate (PyBrOP) and bis(2-oxo-3-oxazolidinyl)phosphinic chloride (BOP-Cl), In some embodiments, the coupling reagent is a uronium. In some embodiments, the uronium coupling reagent is selected from benzotriazole-N,N,N′,N′-tetramethylurea hexafluorophosphate (HBTU), 2-(1H-benzotriazo L-1-yl)-1,1,3,3-Tetramethylurea tetrafluoroborate (TBTU), O-(7-Azabenzotriazol-1-yl)-N,N,N′,N′-tetramethyluronium hexafluorophosphate (HATU), O-(7-Azabenzotriazol-1-yl)-N,N,N′,N′-tetramethyluronium tetrafluoroborate (TATU) and O-(6-Chlorobenzotriazol-1-yl)-N,N,N′,N′-tetramethyluronium hexafluorophosphate (HCTU). In some embodiments, the coupling reagent is an imidazole. In some embodiments, the imidazole coupling reagent is N,N′-carbonyldiimidazole (CDI). In some embodiments, the coupling reagent is selected from pivaloyl chloride (PivCI), oxalyl chloride, isobutyl chloroformate (IBCF), cyanuric chloride (TCT), diphenylphosphinic acid chloride (DppCl), or salt thereof, In one example, the coupling reagent is 1-ethyl-3-(3-dimethylaminopropyl)carbodiimide hydrochloride (EDC·HCl). In one example, the coupling reagent is propyl phosphonic anhydride (T₃P). In one example, the coupling reagent is dicyclohexylcarbodiimide (DCC). In one example, the coupling reagent is diisopropylcarbodiimide (DIC). In one example, the coupling reagent is Hexafluorophosphate Azabenzotriazole Tetramethyl Uronium (HATU). In one example, the coupling reagent is benzotriazole-N,N,N′,N′-tetramethylurea hexafluorophosphate (HBTU). In one example, the coupling reagent is 2-(1H-benzotriazo L-1-yl)-1,1,3,3-Tetramethylurea tetrafluoroborate (TBTU). In one example, the coupling reagent is N,N′-carbonyldiimidazole (CDI). In one example, the coupling reagent is pivaloyl chloride (PivCI). In one example, the coupling reagent is oxalyl chloride. In one example, the coupling reagent is Isobutyl chloroformate (IBCF). In one example, the coupling reagent is cyanuric chloride (TCT). In one example, the coupling reagent is diphenylphosphinic acid chloride (DppCl) In one example, the coupling reagent is 1H-benzotriazol-1-yloxytripyrrolidinyl hexafluorophosphate (PyBOP).

In some embodiments, the coupling reagent is present in an amount of greater than about 0.1, 0.5, 1, 1.5, 2, 2.5, 3, 4, or 5 equivalents, relative to the molar amount of the compound of Formula 4. In some embodiments, the coupling reagent is present in an amount of less than about 10, 9, 8, 7, 6, 5, 4, 3, 2.5, 2, 1.5, 1, or 0.5 equivalents, relative to the molar amount of the compound of Formula 4. In some embodiments, the coupling agent is present in a range provided by any two of these lower and/or upper amounts, such as in a range of between about 0.1 to 10 equivalents, between about 0.5 to 8 equivalents, or between about 1 and 5 equivalents. In some embodiments, the coupling reagent is 1-ethyl-3-(3-dimethylaminopropyl) carbodiimide hydrochloride (EDC·HCl), which can be present in any one of the previously described amounts or ranges thereof. In one example, the coupling reagent is propyl phosphonic anhydride (T₃P), which can be present in any one of the previously described amounts or ranges thereof.

In some embodiments, the process for preparing an amide compound of Formula 2 further comprises an additive. An additive may be any reagent that facilitates/catalyses the amide coupling reaction. Examples of additives include, but are not limited to 2-hydroxypyridine-N-oxide (HOPO), (1-Cyano-2-ethoxy-2-oxoethylidenaminooxy)dimethylamino-morpholino-carbenium hexafluorophosphate (COMU), 1-hydroxybenzotrizole (HOBt), dimethylaminopyridine (DMAP) and any combinations thereof. In one example, the additive is 2-hydroxypyridine-N-oxide (HOPO). In one example, the additive is (1-Cyano-2-ethoxy-2-oxoethylidenaminooxy)dimethylamino-morpholino-carbenium hexafluorophosphate (COMU). In one example, the additive is 1-hydroxybenzotrizole (HOBt). In one example, the additive is dimethylaminopyridine (DMAP).

In some embodiments, the additive is present in an amount of less than about 2.0, about 1.0, about 0.5, about 0.3, about 0.2, or about 0.1 equivalents, relative to the molar amount of the compound of Formula 4. In some embodiments, the additive is present in an amount of greater than about 0.01, about 0.05, about 0.1, about 0.2, about 0.3, about 0.5, or about 1.0 equivalents, relative to the molar amount of the compound of Formula 4. In some embodiments, the additive is present in a range provided by any two of the above upper and/or lower amounts of the additive, such as between 0.01 and 2.0, 0.1 and 0.5, or 0.2 and 0.4. In one example, the additive is HOPO, and is present in an amount of about 0.3 equivalents, relative to the molar amount of the compound of Formula 4.

In some embodiments, the process for preparing an amide compound of Formula 2 further comprises a base. Examples of bases include, but are not limited to N,N-diisopropylethylamine (DIPEA), triethylamine (Et₃N), sodium bicarbonate (NaHCO₃), potassium tert-butoxide (KOtBu), pyridine, lutidine, 1,8-diazabicyclo[5.4.0]undec-7-ene (DBU), sodium methoxide (NaOMe) or N-methylmorpholine (NMM), and any combinations thereof. In one example, the base is N,N-diisopropylethylamine (DIPEA). In one example, the base is triethylamine (Et₃N). In one example, the base is sodium bicarbonate (NaHCO₃). In one example, the base is potassium tert-butoxide (KOtBu). In one example, the base is pyridine. In one example, the base is lutidine. In one example, the base is 1,8-diazabicyclo[5.4.0]undec-7-ene (DBU). In one example, the base is sodium methoxide (NaOMe). In one example, the base is N-methylmorpholine (NMM).

In some embodiments, the base is present in an amount of less than about 10, 5, 4, 3, 2, 1, 0.5, or 0.1 equivalents, relative to the molar amount of the compound of Formula 4. In some embodiments, the base is present in an amount of greater than about 0.1, 0.2, 0.3, 0.5, 0.6, 0.8, 1.0, 1.2, or 1.4. equivalents, relative to the molar amount of the compound of Formula 4. In some embodiments, the base is present in a range provided by any two of the above upper and/or lower amounts of the additive, such as between 0.1 and 7, 0.5 and 3, or 1 and 2. In one example, the base is DIPEA, and is present in any one of the previously described amounts or ranges thereof.

In some embodiments, the process for preparing an amide compound of Formula 2 is performed in the presence of a catalyst. As used herein, the term “catalyst” refers to a compound that promotes or increases the rate of a reaction between compounds without itself being consumed. In one embodiment, the catalyst promotes or increases the rate of reaction between a compound of Formula 4 and a compound of Formula 5 to prepare an amide compound of Formula 2. In one embodiment, the catalyst for promoting or increasing the rate of reaction between a compound of Formula 4 and a compound of Formula 5 to prepare an amide compound of Formula 2 is a boron compound, or salt thereof. In one example, the catalyst for promoting or increasing the rate of reaction between a compound of Formula 4 and a compound of Formula 5 to prepare an amide compound of Formula 2 is boric acid. In one example, the catalyst for promoting or increasing the rate of reaction between a compound of Formula 4 and a compound of Formula 5 to prepare an amide compound of Formula 2 is phenylboronic acid. In one example, the catalyst for promoting or increasing the rate of reaction between a compound of Formula 4 and a compound of Formula 5 to prepare an amide compound of Formula 2 is 2-bromophenylboronic acid. In one example, the catalyst for promoting or increasing the rate of reaction between a compound of Formula 4 and a compound of Formula 5 to prepare an amide compound of Formula 2 is borazine (ammonia borane; BH₃·NH₃).

In some embodiments, the process for preparing an amide compound of Formula 2 is performed in a suitable solvent. In some embodiments, the process for preparing an amide compound of Formula 2 is performed in a polar protic solvent. Examples of polar protic solvents include, but are not limited to, alcohols, glycols, and any combinations thereof. Examples of alcohols include, but are not limited to, methanol (MeOH), ethanol (EtOH), 1-propanol, isopropyl alcohol (2-propanol, iPrOH or IPA), 1-butanol, 2-butanol, 1-butanol (1-BuOH), 1-pentanol, 3-methyl-1-butanol, 2-methyl-1-propanol, and combinations thereof. Examples of glycols include, but are not limited to, ethylene glycol. In some embodiments, the process for preparing an amide compound of Formula 2 is performed in a polar aprotic solvent. In some embodiments, the process for preparing an amide compound of Formula 2 is performed in a non-polar ether solvent. In some embodiments, the process for preparing an amide compound of Formula 2 is performed in a polar or non-polar chlorocarbon solvent. In some embodiments, the process for preparing an amide compound of Formula 2 is performed in a non-polar hydrocarbon or aromatic hydrocarbon solvent. In some embodiments, the process for preparing an amide compound of Formula 2 is performed in any combination of polar protic, polar aprotic, non-polar ether, chlorocarbon or non-polar solvents. Examples of polar aprotic solvents include, but are not limited to, ketones, nitriles, esters, carbonate esters, ethers, sulfoxides, sulfones, amides, nitroalkanes, pyrrolidines, pyridines, and combinations thereof. Examples of ketones include, but are not limited to, acetone, methylethyl ketone (MEK), methylbutyl ketone (MBK), methylisobutyl ketone (MIBK), methylisopropyl ketone (MIPK) and combinations thereof. Examples of nitriles include, but are not limited to, acetonitrile (MeCN). Examples of esters include, but are not limited to, ethyl formate, methyl acetate (MeOAc), ethyl acetate (EtOAc), propyl acetate, isopropyl acetate (iPAC), n-butyl acetate, and isobutyl acetate, and combinations thereof. Examples of carbonate esters include, but are not limited to, dimethyl carbonate (DMC), propylene carbonate (PC), and combinations thereof. Examples of polar and non-polar ethers include, but are not limited to, methyl-tert-butyl ether (MTBE), diethyl ether, 1,4-dioxane, 2-methoxyethanol, 2-ethoxyethanol, 1,2-dimethoxyethane (DME or monoglyme), 1,1-dimethoxymethane, 2,2-dimethoxypropane, 1,1-diethoxypropane, isopropyl ether, petroleum ether, cyclopentyl methyl ether (CPME), anisole (methoxybenzene), methyltetrahydrofuran (MeTHF), and tetrahydrofuran (THF), and combinations thereof. Examples of sulfoxides include, but are not limited to, dimethylsulfoxide (DMSO). Examples of sulfones include, but are not limited to, sulfolane. Examples of amides include, but are not limited to, formamide, N,N-dimethylacetamide, and N,N-dimethylformamide (DMF), and combinations thereof. Examples of nitroalkanes include, but are not limited to, nitromethane. Examples of pyrrolidines include, but are not limited to, N-methylpyrrolidone (NMP). Examples of polar and non-polar chlorocarbons include, but are not limited to, dichloromethane (DCM), chloroform, 1,2-dichloroethane, 1,1,1-trichloroethane, 1,1-dichloroethene, 1,2-dichloroethene, and combinations thereof. In one example, the polar aprotic solvent is ethyl acetate (EtOAc). In one example, the polar aprotic solvent is acetonitrile (MeCN). In one example, the polar aprotic solvent is N-methyl-2-pyrrolidone (NMP). In one example, the polar aprotic solvent is methyl-tert-butyl ether (MTBE). In one example, the polar aprotic solvent is N-methyl-2-pyrrolidone (NMP) in combination with methyl-tert-butyl ether (MTBE). In one example, the polar aprotic solvent is N,N-dimethylformamide (DMF). In one example, the polar aprotic solvent is pyridine. In one example, the polar aprotic solvent is acetone. In one example, the polar aprotic solvent is methyltetrahydrofuran (MeTHF). In one example, the polar aprotic solvent is 1,4-dioxane. In one example, the polar aprotic solvent is dimethylsulfoxide (DMSO). In one example, the polar chlorocarbon solvent is dichloromethane (DCM). Examples of non-polar solvents include, but are not limited to, toluene, hexanes, n-heptane, cyclohexane, octane, isooctane, cyclopentane, benzene, and xylenes, or combinations thereof. In one example, the non-polar solvent is toluene. In one example, the non-polar solvent is a xylene. In one example, the non-polar solvent is hexanes. In one example, the non-polar solvent is cyclohexane. In one example, the non-polar solvent is n-heptane.

The person skilled in the art will further appreciate that it may be advantageous to apply heat to facilitate the reaction. The amount of heat required may depend upon the solvent in which the reaction is performed in, as discussed above. In one example, the process for preparing a compound of Formula 2 is performed in the presence of heat. In some embodiments, the process is performed at a temperature (in ° C.) of at least about 10, 20, 30, 40, 50, 60, 70, 80, or 90. In some embodiments, the process is performed at a temperature (in ° C.) of less than about 100, 90, 80, 70, 60, 50, 40, 60, or 20. The temperature may be present in a range provided by any two of these upper and/or lower values, for example between about 10° C. to 50° C., 15° C. to 40° C. or 20° C. to 30° C. In one example, the process for preparing an amide compound of Formula 2 is performed in the presence of heat. In one example, the process for preparing an amide compound of Formula 2 is performed at about room temperature.

The compound of Formula 2 may be formed as a salt. The nature of the salt formed in the process will be dependent upon the reagents used in the process for preparing an amide compound of Formula 2. The compound of Formula 2 may be isolated prior to a following reaction. In one example, the compound of Formula 2 is isolated prior to the following reaction. Alternatively, the compound of Formula 2 may be reacted in a following reaction without isolation and/or purification (i.e., reacted in situ). That is, the compound of Formula 2 may be reacted in a following reaction in crude form. In one example, the compound of Formula 2 is reacted in a following reaction without isolation and/or purification.

The starting ester compound of Formula 4, or salt thereof, preferably has a low content of water present before using it in the amide coupling with a compound of Formula 5. In some embodiments, the isolated product or solution of the compound of Formula 4, or salt thereof, is dried to further remove water that may be present. In some embodiments, the water content present as a wt % of the compound of Formula 4 is less than about 5, 4, 3, 2, 1, 0.75, 0.5, 0.25, 0.1, 0.05, or 0.01.

The starting ester compound of Formula 4, or salt thereof, preferably has a low content of residual solvent remaining from the acid-catalysed esterification reaction before using it in the amide coupling with a compound of Formula 5. In some embodiments, the isolated product or solution of the compound of Formula 4, or salt thereof, is dried to further remove residual reaction solvent that may be present. In some embodiments, the reaction solvent content present as a wt % of the compound of Formula 4 is less than about 5, 4, 3, 2, 1, 0.75, 0.5, 0.25, 0.1, 0.05, or 0.01.

### Synthesis of a Compound of Formula 4

In some embodiments, there is provided a process for preparing a compound of Formula 4, or salt thereof:

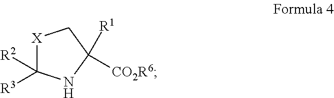

comprises reacting a compound of Formula 6, or salt thereof:

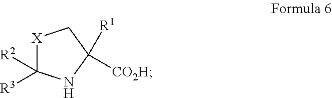

under acid-catalysed esterification conditions.

In some embodiments, a process is provided for preparing a compound of Formula 4 or salt thereof:

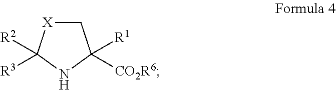

comprising reacting a compound of Formula 7:

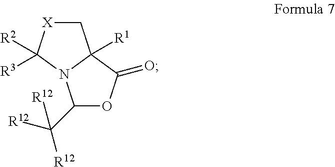

under acid-catalysed esterification conditions.

In some embodiments, X is selected from CR⁷R⁸, NR⁷, O, and S, as described herein. In some embodiments, and R⁷ and R⁸ are each independently selected from hydrogen and alkyl, as described herein. In some embodiments, R¹, R², and R³ are each independently selected from hydrogen, halogen, alkyl, alkenyl, alkynyl, haloalkyl, —O-haloalkyl, 3-10-membered carbocycle, 3-10-membered heterocycle, —OR⁹, —SR⁹, —NR⁹R¹⁰, —NO₂, —CN, —C(O)R⁹, —C(O)OR⁹, —C(O)NR⁹R¹⁰, and —CNR⁹, as described herein. In some embodiments, R⁹ and R¹⁰ are each independently selected from hydrogen, —C₁₋₈alkyl, and —C₂₋₈alkenyl, as described herein. In some embodiments, R⁶ is selected from alkyl, aryl and alkylaryl, as described herein. In one example, R⁶ is —CH₃. In one example, R⁶ is —CH₂CH₃. In one example, R⁶ is aryl. In one example, R⁶ is phenyl. In one example, R⁶ is alkylaryl. In one example, R⁶ is benzyl. In some embodiments, R¹² is selected from chlorine or methyl (—CH₃). In one example, R¹² is chlorine. In one example, R¹² is methyl (—CH₃).

Acid-catalysed esterification conditions will be understood to include any suitable reaction conditions that serve to install an ester group (i.e., conversion of a carboxylic acid group to an ester group). In particular, acid-catalysed esterification conditions include any suitable reaction conditions that convert the carboxylic acid of Formula 6 to the ester of Formula 4. In some embodiments, the acid-catalysed esterification conditions include the presence of an acid and a methyl source, such that the methyl ester is formed (i.e., R⁶ is —CH₃). In one example, the methyl source is methanol. That is, the reaction is undertaken in methanol. In some embodiments, the acid-catalysed esterification conditions include an acid and methanol. In some embodiments, the acid-catalysed esterification conditions include the presence of an acid and an ethyl source, such as ethanol so that the ethyl ester is formed (i.e., R⁶ is —CH₂CH₃). In some embodiments, the acid-catalysed esterification conditions include the presence of an acid and a benzyl source, such as benzyl alcohol so that the benzyl ester is formed (i.e., R⁶ is benzyl).

The reagent utilised in the acid-catalysed esterification conditions includes any reagent that catalyses/promotes the reaction to install an ester group. In some embodiments, the reagent is an acid, for example selected from hydrogen chloride and/or a sulphonic acid. In other examples, the reagent is selected from hydrogen chloride, methanolic hydrogen chloride, methanesulfonic acid, sulfuric acid, thionyl chloride, p-toluenesulfonic acid, acetyl chloride, trimethylsilyl chloride, and oxalyl chloride. In one example, the reagent is hydrogen chloride. In one example, the reagent is thionyl chloride. In one example, the reagent is methanesulfonic acid. In one example, the reagent is oxalyl chloride. In one example, the reagent is trimethylsilyl chloride. In one example, the acid is acetyl chloride. In one example, the acid is sulfuric acid. In some embodiments, the reagent is present in an amount of at least about 1, at least about 2, at least about 3, at least about 4, at least about 5, at least about 6, at least about 7, at least about 8, at least about 9, or at least about 10 equivalents, relative to the molar amount of the compound of Formula 6. In some embodiments, the reagent is present in an amount of less than about 9, less than about 8, less than about 7, less than about 6, less than about 5, or less than about 4 equivalents, relative to the molar amount of the compound of Formula 6. In some embodiments, the reagent is present in a range provided by any two of the above upper and/or lower amounts of the reagent, such as between 1 and 10, 2 and 8, or 3 and 6.

In some embodiments, the acid is present at a concentration of less than about 10 N, 9 N, 8 N, 7 N, 6 N, 5 N, or 4 N. In some embodiments, the acid is present at a concentration of at least about 1 N, 2 N, 3 N, 4 N, or 5 N. In some embodiments, the acid is present at a concentration range provided by any two of the upper and/or lower values, for example between about 1 N and 10 N, between about 2 N and 9 N, or between about 3 N and 6 N.

Depending on the reagent used, it may be preferable to minimise the water present in the process. That is, reducing the water present in the process may result in a reduced reaction time and/or greater conversion to the ester product of Formula 4.

Again, as will be appreciated by the person skilled in the art, it may be necessary to apply heat to facilitate the reaction. The amount of heat required may depend upon the solvent in which the reaction is performed in, as discussed above. In one example, the process for preparing a compound of Formula 4 is performed in the presence of heat. In some embodiments, the process is performed at a temperature of between about 10° C. to 100° C., 20° C. to 90° C., 30° C. to 80° C., about 40° C. to 70° C., or between about 50° C. and 60° C. In one example, the process for preparing an ester product of Formula 4 is performed at a temperature of between about 50° C. and 65° C.

The compound of Formula 4 may be formed as a salt. The nature of the salt formed in the process will be dependent upon the reagents used in the acid-catalysed esterification, as would be understood by the person skilled in the art. In one example, the compound of Formula 4 is formed as the hydrochloride salt. The esterified product, the compound of Formula 4, may be isolated prior to a subsequent reaction. In one example, the compound of Formula 4, is isolated prior to the subsequent reaction. Alternatively, the esterified product, a compound of Formula 4, may be reacted in a subsequent reaction without isolation and/or purification (i.e., reacted in situ). In one example, the esterified product, the compound of Formula 4, is reacted in a subsequent reaction without isolation and/or purification.

The compound of Formula 4 may be obtained as a salt that is isolated in crystalline form. In one example, a compound of Formula 4 is isolated as the crystalline hydrochloric acid salt. In one example, a compound of Formula 4 is isolated as the crystalline mesylate salt. In one example, a compound of Formula 4 is isolated as the crystalline sulfate salt.

Alternatively, the compound of Formula 4 may be extracted as the free-base of the ester from an aqueous basic solution into an organic solvent. In one example, the compound of Formula 4 is extracted as the free-base of the methyl ester from an aqueous potassium carbonate (K₂CO₃) solution into an organic solvent. In one example, the compound of Formula 4 is extracted as the free-base of the methyl ester from an aqueous potassium carbonate solution into methyl-tert-butyl ether (MTBE) solvent.

When the ester compound of Formula 4 is extracted as the free-base into a solvent, it may be preferable to minimise the amount of water present in the solution before using it in the amide coupling of a compound of Formula 4 with a compound of Formula 5. In some embodiments, water produced as a by-product is azeotropically dried. In one example, the water content of a solution of the compound of Formula 4 as the free-base of the ester is reduced to low levels by azeotropically drying.

In some embodiments, the water content in the extracted solution after preparing the compound of Formula 4 (wt % of total solution) is less than about 5, 4, 3, 2, 1, 0.75, 0.5, 0.25, 0.1, 0.05, or 0.01. In one example, the water content in the extracted solution after preparing the compound of Formula 4 (wt % of total solution) is less than about 1 wt %. In some embodiments, the water content in the solids after solvent removal in preparing the compound of Formula 4 (wt % of total solids) is less than about 5, 4, 3, 2, 1, 0.75, 0.5, 0.25, 0.1, 0.05, or 0.01. In some embodiments, the water content in the solids after isolation or purification in preparing the compound of Formula 4 (wt % of total solids) is less than about 5, 4, 3, 2, 1, 0.75, 0.5, 0.25, 0.1, 0.05, or 0.01.

When the compound of Formula 4 is extracted as the free-base of the ester into a solvent, it may be preferable to minimise the reaction solvent remaining in the solution before using it in the amide coupling of a compound of Formula 4 with a compound of Formula 5. In some embodiments, residual reaction solvent is azeotropically dried. In one example, the reaction solvent content of a solution of the compound of Formula 4 as the free-base of the ester is reduced to low levels by azeotropically drying.

In some embodiments, the reaction solvent in the extracted solution after preparing the compound of Formula 4 (wt % of total solution) is less than about 5, 4, 3, 2, 1, 0.75, 0.5, 0.25, 0.1, 0.05, or 0.01. In one example, the reaction solvent in the extracted solution after preparing the compound of Formula 4 (wt % of total solution) is less than about 0.5 wt %. In some embodiments, the reaction solvent in the solids after solvent removal in preparing the compound of Formula 4 (wt % of total solids) is less than about 5, 4, 3, 2, 1, 0.75, 0.5, 0.25, 0.1, 0.05, or 0.01. In some embodiments, the reaction solvent in the solids after isolation or purification in preparing the compound of Formula 4 (wt % of total solids) is less than about 5, 4, 3, 2, 1, 0.75, 0.5, 0.25, 0.1, 0.05, or 0.01. In one example, the reaction solvent is methanol.

In other embodiments, there is provided one or more processes for preparing a compound of Formula 1 from any one or more of the above aspects, embodiments, or examples thereof. In other embodiments, there is provided a compound of Formula 1 or Formula 4 prepared from any one or more processes as described by the above aspects, embodiments, or examples thereof.

### Synthesis of a Compound of Formula 7

In another aspect or embodiment, there is provided a process for preparing a compound of Formula 7:

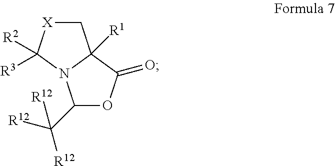

comprising reacting a compound of Formula 8:

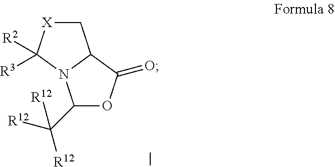

with a compound of Formula 9:

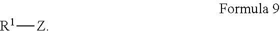

In some embodiments, X is selected from CR⁷R⁸, NR⁷, O, and S, or according to any embodiments or examples thereof as described herein. In some embodiments, R⁷ and R⁸ are each independently selected from hydrogen and alkyl, or according to any embodiments or examples thereof as described herein.

In some embodiments, R², and R³ are each independently selected from hydrogen, halogen, alkyl, alkenyl, alkynyl, haloalkyl, —O-haloalkyl, 3-10-membered carbocycle, 3-10-membered heterocycle, —OR⁹, —SR⁹, —NR⁹R¹⁰, —NO₂, —CN, —C(O)R⁹, —C(O)OR⁹, —C(O)NR⁹R¹⁰, and —CNR⁹, as described herein. In some embodiments, R⁹ and R¹⁰ are each independently selected from hydrogen, —C₁₋₈alkyl, and —C₂₋₈alkenyl, as described herein.

In some embodiments, R¹ is selected from alkyl, alkenyl, alkynyl, haloalkyl, 3-10-membered carbocycle, and 3-10-membered heterocycle. In some embodiments, R¹ is selected from alkyl and alkenyl. In some embodiments, R¹ is independently selected from C₁₋₈alkyl, and C₂₋₈alkenyl. In some embodiments, R¹ is selected from an alkenyl group. In one example, R¹ is a methyl group. In one example, R¹ is an allyl group.

In some embodiments, R¹² is selected from chlorine and methyl (—CH₃). In one example, R¹² is chlorine. In one example, R¹² is methyl (—CH₃).

In some embodiments, Z is a leaving group. Suitable leaving groups include, but are not limited to, halogens. In one example, Z is a halogen. In some embodiments, Z is selected from iodine, bromine, and chlorine. In one example, Z is iodine. In one example, Z is bromine. In one example, Z is chlorine.

In some embodiments, the process for preparing a compound of Formula 7 occurs in the presence of a strong base. As would be understood by the person skilled in the art, a strong base will completely dissociate in an aqueous solution. In some embodiments, the base is a Lewis base, and any combination thereof. Such bases may also be referred to as “superbases”. Examples of Lewis bases include, but are not limited to, lithium diisopropylamide (LDA), n-butyllithium (n-BuLi), lithium diethylamide (LDEA), sodium amide (NaNH₂), sodium hydride (NaH), and lithium bis(trimethylsilyl)amide (lithium hexamethyldisilazide or LiHMDS). In some embodiments, the strong base is selected from lithium diisopropylamide (LDA) and n-butyllithium (n-BuLi). In one example, the strong base is lithium diisopropylamide (LDA). In one example, the strong base is n-butyllithium (n-BuLi).

In some embodiments, the process for preparing a compound of Formula 7 is performed in a suitable solvent. In some embodiments, the process for preparing a compound of Formula 7 is performed in a polar aprotic solvent. In some embodiments, the process for preparing a compound of Formula 7 is performed in a non-polar ether solvent. Examples of polar aprotic solvents include, but are not limited to, ethers, sulfoxides, amides, amines, ureas, and combinations thereof. Examples of polar and non-polar ethers include, but are not limited to, methyl-tert-butyl ether (MTBE), diethyl ether, dimethyl ether, 1,4-dioxane, bis(2-methoxyethyl) ether (diglyme), 1,2-dimethoxyethane (DME or monoglyme), 1,1-dimethoxymethane, 2,2-dimethoxypropane, 1,1-diethoxypropane, isopropyl ether, cyclopentyl methyl ether (CPME), anisole (methoxybenzene), methyltetrahydrofuran (MeTHF), and tetrahydrofuran (THF), and combinations thereof. Examples of sulfoxides include, but are not limited to, dimethylsulfoxide (DMSO). Examples of amides include, but are not limited to, formamide, N,N-dimethylacetamide, N,N-dimethylformamide (DMF), and combinations thereof. Examples of amines include, but are not limited to, ammonia, tetramethylethylenediamine (TMEDA), and combinations thereof. Examples of ureas include, but are not limited to, N,N-dimethylpropyleneurea (DMPU). In some embodiments, the process for preparing a compound of Formula 7 is performed in a non-polar hydrocarbon solvent. In some embodiments, the process for preparing a compound of Formula 7 is performed in any combination of a non-polar ether or hydrocarbon solvents. Examples of non-polar hydrocarbon solvents include, but are not limited to, alkanes, cycloalkanes, and aromatic hydrocarbons. Examples of alkanes include, but are not limited to, pentanes, hexanes, heptanes, isooctane, and combinations thereof. Examples of cycloalkanes include, but are not limited to, cyclohexane, methylcyclohexane, and combinations thereof. Examples of aromatic hydrocarbons include, but are not limited to, benzene, ethylbenzene, toluene, m-xylene (m-dimethylbenzene), cumene (isopropylbenzene), and combinations thereof. In one example, the aromatic hydrocarbon solvent is ethylbenzene. In one example, the hydrocarbon solvent is a combination of hexanes and ethylbenzene. In one example, the hydrocarbon solvent is a combination of heptane, ethylbenzene, and tetrahydrofuran (THF). In one example, the ether solvent is tetrahydrofuran (THF).

To further reduce introduction of impurities, the reaction can be performed at a reduced temperature (i.e., a temperature below room temperature). In some embodiments, the process for preparing a compound of Formula 7 is conducted at a reduced temperature. In some embodiments, the process for preparing a compound of Formula 7 is performed in the temperature range of about −90° C. to about 0° C., −80° C. to about −5° C., −70° C. to about −10° C., about −50° C. to about −20° C., or about −40° C. to about −30° C. In one example, the process for preparing a compound of Formula 7 is performed in the temperature range of about −40° C. to about −30° C. In some embodiments, the reaction involves a deprotonation and then alkylation, wherein the deprotonation is conducted at a temperature of between about −90° C. to about-40° C., about −85° C. to about −50° C., or about −80° C. to about −65° C., and the alkylation is conducted at a temperature of about −90° C. to about 0° C., or about −70° C. to −5° C.

In other embodiments, there is provided one or more processes for preparing a compound of Formula 1 from any one or more of the above aspects, embodiments, or examples thereof. In other embodiments, there is provided a compound of Formula 1 or Formula 7 prepared from any one or more processes as described by the above aspects, embodiments, or examples thereof.

### Overall Synthesis of a Bicyclic Glycine-Proline Compound of Formula 1

Provided herein is an example overall process for preparing a bicyclic glycine-proline compound of Formula 1 as depicted in Scheme 3 below:

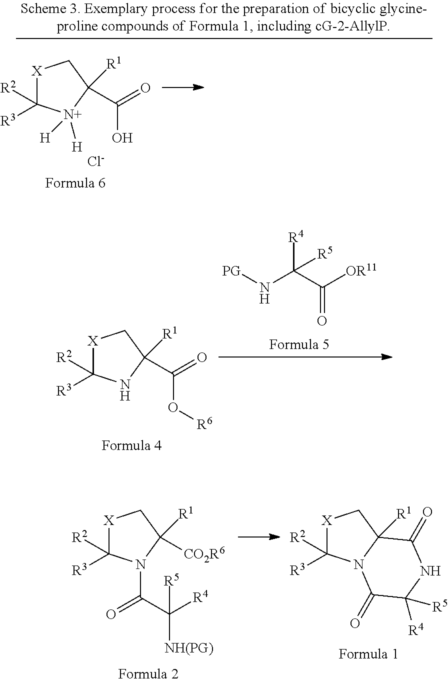

Accordingly, in some embodiments, there is provided a process for preparing a bicyclic glycine-proline compound of Formula 1:

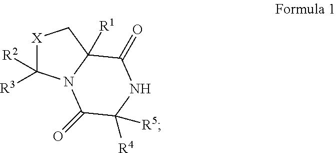

comprising the steps of:
i) preparing a compound of Formula 2 according to any embodiments or examples thereof as described herein:

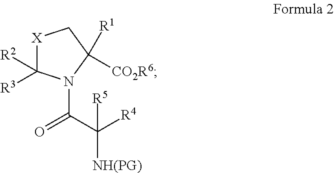

comprising reacting a compound of Formula 4 or salt thereof:

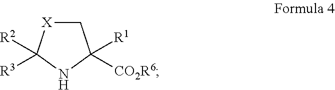

with a compound of Formula 5:

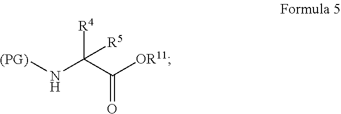

under amide coupling conditions; and
ii) performing a base initiated cyclisation reaction of a compound of Formula 2 to form the compound of Formula 1 according to any embodiments or examples thereof as described herein:

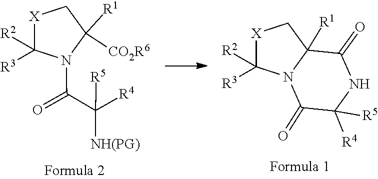

wherein
X is selected from CR⁷R⁸, NR⁷, O, and S;
R⁷ and R⁸ are each independently selected from hydrogen and alkyl;
R¹, R², R³, R⁴, and R⁵ are each independently selected from hydrogen, halogen, alkyl, alkenyl, alkynyl, haloalkyl, —O-haloalkyl, 3-10-membered carbocycle, 3-10-membered heterocycle, —OR⁹, —SR⁹, —NR⁹R¹⁰, —NO₂, —CN, —C(O)R⁹, —C(O)OR⁹, —C(O)NR⁹R¹⁰, and —CNR⁹, or R⁴ and R⁵ taken together is a 3-10-membered carbocycle; and R⁹ and R¹⁰ are each independently selected from hydrogen, —C₁₋₈alkyl, and —C₂₋₈alkenyl;
R⁶ is selected from alkyl, aryl and alkylaryl;
PG is an amine protecting group;
R¹¹ is selected from hydrogen and AG; and
AG is an activating group.

In some embodiments, there is also provided a process for preparing a bicyclic glycine-proline compound of Formula 1a:

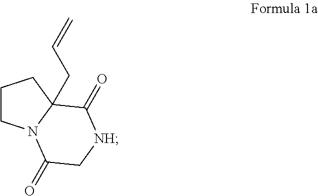

comprising the steps of:
i) preparing a compound of Formula 2a according to any embodiments or examples thereof as described herein:

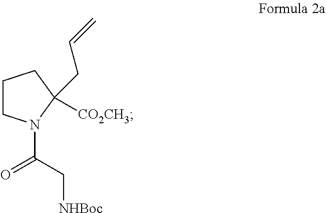 [SMILES: C=CCC1(C(=O)OC)CCCN1C(=O)CNC(=O)OC(C)(C)C]

comprising reacting a compound of Formula 4a or salt thereof:

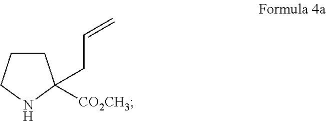

with a compound of Formula 5a:

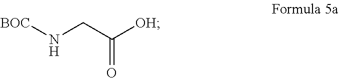 [SMILES: CC(C)(C)OC(=O)NCC(=O)O]

under amide coupling conditions; and
ii) performing a base initiated cyclisation reaction of a compound of Formula 2a to form the compound of Formula 1a according to any embodiments or examples thereof as described herein:

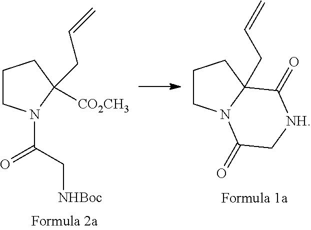 [SMILES: C=CCC1(C(=O)OC)CCCN1C(=O)CNC(=O)OC(C)(C)C ; C=CCC12CCCN1C(=O)CNC2=O]

In some embodiments, there is also provided a process for preparing a bicyclic glycine-proline compound of Formula 1a(R):

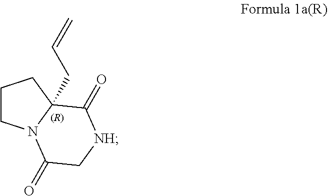

comprising the steps of:
i) preparing a compound of Formula 2a(R) according to any embodiments or examples thereof as described herein:

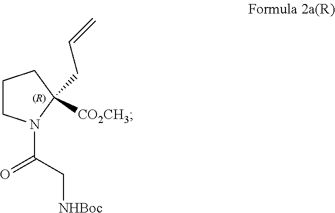

comprising reacting a compound of Formula 4a(R) or salt thereof:

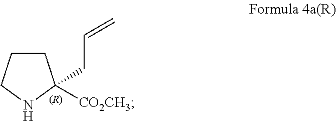

with a compound of Formula 5a:

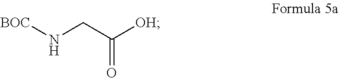 [SMILES: CC(C)(C)OC(=O)NCC(=O)O]

under amide coupling conditions; and
ii) performing a base initiated cyclisation reaction of a compound of Formula 2a(R) to form the compound of Formula 1a(R) according to any embodiments or examples thereof as described herein:

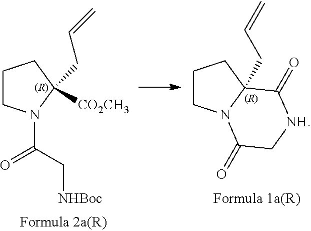

In some embodiments, there is also provided a process for preparing a bicyclic glycine-proline compound of Formula 1a(S):

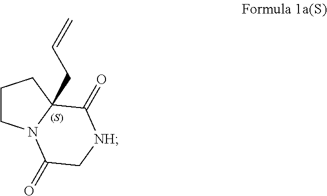

comprising the steps of:
i) preparing a compound of Formula 2a(R) according to any embodiments or examples thereof as described herein:

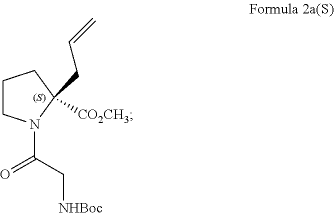

comprising reacting a compound of Formula 4a(S) or salt thereof:

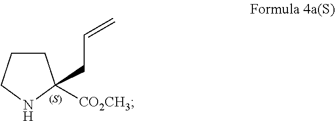

with a compound of Formula 5a:

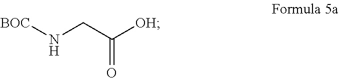 [SMILES: CC(C)(C)OC(=O)NCC(=O)O]

under amide coupling conditions; and
ii) performing a base initiated cyclisation reaction of a compound of Formula 2a(S) to form the compound of Formula 1a(S) according to any embodiments or examples thereof as described herein:

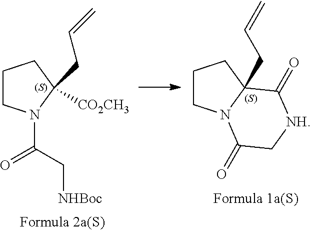

In some embodiments, there is provided a process for preparing a bicyclic glycine-proline compound of Formula 1:

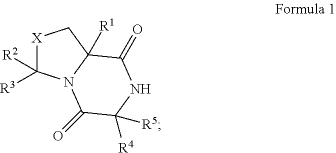

comprising the steps of:
i) preparing a compound of Formula 4 or salt thereof, according to any embodiments or examples thereof as described herein:

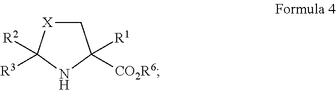

comprising reacting a compound of Formula 6 or salt thereof:

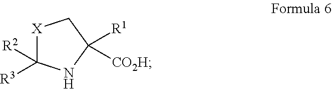

under acid-catalysed esterification conditions;
ii) preparing a compound of Formula 2 according to any embodiments or examples thereof as described herein:

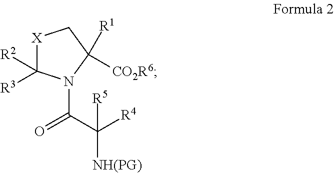

comprising reacting a compound of Formula 4 or salt thereof:

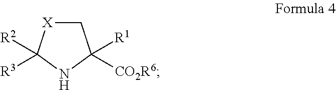

with a compound of Formula 5:

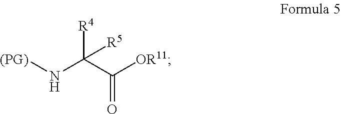

under amide coupling conditions; and
iii) performing a base initiated cyclisation reaction of a compound of Formula 2 to form the compound of Formula 1 according to any embodiments or examples thereof as described herein:

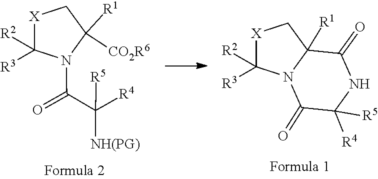

wherein
X is selected from CR⁷R⁸, NR⁷, O, and S;
R⁷ and R⁸ are each independently selected from hydrogen and alkyl;
R¹, R², R³, R⁴, and R⁵ are each independently selected from hydrogen, halogen, alkyl, alkenyl, alkynyl, haloalkyl, —O-haloalkyl, 3-10-membered carbocycle, 3-10-membered heterocycle, —OR⁹, —SR⁹, —NR⁹R¹⁰, —NO₂, —CN, —C(O)R⁹, —C(O)OR⁹, —C(O)NR⁹R¹⁰, and —CNR⁹, or R⁴ and R⁵ taken together is a 3-10-membered carbocycle; and R⁹ and R¹⁰ are each independently selected from hydrogen, —C₁₋₈alkyl, and —C₂₋₈alkenyl;
R⁶ is selected from alkyl, aryl and alkylaryl;
PG is an amine protecting group;
R¹¹ is selected from hydrogen and AG.

It will be appreciated that the above process for preparing a compound of Formula 1 may also be provided for preparing any stereoisomer compound of Formula 1 using an appropriate stereoisomer compound of any one or more of Formula 6, Formula 5, Formula 4, and Formula 2, according to any embodiments or examples thereof as described herein.

### Scale-up Process

The process, as described herein, allows for the scalable synthetic pathway for the manufacture of a bicyclic glycine-proline compound of Formula 1. The process as described herein, for example when compared to the process described in international patent application WO2005023815, can provide increased overall yield and purity of a compound of Formula 1 under scalable reaction conditions.

In some embodiments, the process is conducted on small-scale (e.g., scale of 20 mg to 1 gram), as would be suitable for research and development purposes. However, in some other embodiments, the process is conducted on large-scale (e.g., scale of 1 kg to 500 kg), as would be suitable for manufacturing purposes. The synthesis or one or more steps thereof may occur as a batch-type process.

In some embodiments, the process for preparation of a bicyclic glycine-proline compound of Formula 1 occurs with a starting material amount of a compound of Formula 2 of at least 1 g, at least 10 g, at least 50 g, at least 100 g, at least 500 g, at least 1 kg, at least 10 kg, at least 25 kg, at least 50 kg, at least 75 kg, or at least 100 kg. That is, the process for preparing a bicyclic glycine-proline compound of Formula 1 occurs on at least 1 g, at least 10 g, at least 50 g, at least 100 g, at least 500 g, at least 1 kg, at least 10 kg, at least 25 kg, at least 50 kg, at least 75 kg, or at least 100 kg scale. In one example, the process for preparing a bicyclic glycine-proline compound of Formula 1a occurs on at least 1 g, at least 10 g, at least 50 g, at least 100 g, at least 500 g, at least 1 kg, at least 10 kg, at least 25 kg, at least 50 kg, at least 75 kg, or at least 100 kg scale. In one example, the process for preparing a bicyclic glycine-proline compound of Formula 1a(R) occurs on at least 1 g, at least 10 g, at least 50 g, at least 100 g, at least 500 g, at least 1 kg, at least 10 kg, at least 25 kg, at least 50 kg, at least 75 kg, or at least 100 kg scale. In one example, the process for preparing a compound of Formula 2 occurs with a starting material amount of a compound of Formula 4 of at least 1 g, at least 10 g, at least 50 g, at least 100 g, at least 500 g, at least 1 kg, at least 10 kg, at least 20 kg, at least 30 kg, or at least 40 kg scale. In one example, the process for preparing a compound of Formula 4 occurs with a starting material amount of a compound of Formula 6 of at least 1 g, at least 10 g, at least 50 g, at least 100 g, at least 500 g, at least 1 kg, at least 10 kg, at least 25 kg, or at least 50 kg scale.

In some embodiments, the process provides a molar conversion of a compound of Formula 2 to a bicyclic glycine-proline compound of Formula 1 of at least 40%, at least 50%, at least 60%, at least 70%, at least 75%, at least 80%, at least 90%, at least 95%, or at least 98%, as measured by HPLC. It will be understood that the conversion of a reaction may be measured at any point during the reaction, through any suitable technique, such as TLC or HPLC. Typically, an aliquot of the reaction mixture will be subject to HPLC, where the relevant component peaks are identified and integrated relative to one another. In some embodiments, the process of a cyclisation reaction of a compound of Formula 2 to form a protected bicyclic compound of Formula 3, and then removing the protecting group from the compound of Formula 3 to form the bicyclic glycine-proline compound of Formula 1, provides a molar conversion of a compound of Formula 2 to a compound of Formula 1 of at least 40%, at least 50%, at least 60%, at least 70%, at least 75%, at least 80%, at least 90%, at least 95%, or at least 98%, as measured by HPLC.

As used herein, the term “yield” will be taken to mean the amount of either crude or purified compound obtained from a reaction, measured as a percentage of theoretical yield of the compound in that reaction, as would be understood by the person skilled in the art.

In some embodiments, the process provides a yield of a bicyclic glycine-proline compound of Formula 1 of at least 20%, at least 30%, at least 40%, at least 50%, at least 60%, at least 70%, at least 80%, at least 90%, or at least 95%, as determined from a compound of Formula 2 starting material. That is, in some embodiments, the process of a cyclisation reaction of a compound of Formula 2 to form a protected bicyclic glycine-proline compound of Formula 3, and then removing the protecting group from the compound of Formula 3 to form the bicyclic glycine-proline compound of Formula 1, as described herein, provides at least 20%, at least 30%, at least 40%, at least 50%, at least 60%, at least 70%, at least 80%, at least 90%, or at least 95%, yield of a bicyclic glycine-proline compound of Formula 1. In some embodiments, the process of reacting a compound of Formula 2 to form the cyclic compound of Formula 1, as described herein, can provide between about 50% and 95%, between about 70% and 95%, or between about 80% and 95% yield of a bicyclic glycine-proline compound of Formula 1.

In some embodiments, the process described herein provides a bicyclic glycine-proline compound of Formula 1 in high purity. As would be understood by a skilled person, purity is a measure independent of yield. That is, a compound may have a high purity, albeit a low yield. As used herein, the term “high purity” refers to at least 80% of the ultimately obtained material being the desired compound (e.g., Formula 1), which may be measured, for example, by HPLC methods. The purity of a compound may be measured based on the crude reaction mixture, the product isolated from the reaction mixture (i.e., following the reaction work-up), or the purified product (i.e., following chromatography, recrystallization, etc.).

In some embodiments, the process of reacting a compound of Formula 2 to form the bicyclic glycine-proline compound of Formula 1, as described herein, provides a bicyclic glycine-proline compound of Formula 1 in at least 80%, at least 85%, at least 90%, at least 95%, at least 96%, at least 97%, at least 98%, at least 99%, at least 99.5%, at least 99.8%, or at least 99.9% purity. Crystallisation can provide a purity of about 100%. In one example, the process of reacting a compound of Formula 2 to form the bicyclic glycine-proline compound of Formula 1, as described herein, provides a bicyclic glycine-proline compound of Formula 1 in at least 80%, at least 85%, at least 90%, at least 95%, at least 98%, at least 99%, at least 99.5%, at least 99.8%, or at least 99.9% purity of the product in the crude reaction mixture. In one example, the process of reacting a compound of Formula 2 to form the bicyclic glycine-proline compound of Formula 1, as described herein, provides a bicyclic glycine-proline compound of Formula 1 in at least 80%, at least 85%, at least 90%, at least 95%, at least 98%, at least 99%, at least 99.5%, at least 99.8%, or at least 99.9% purity of the product isolated from the reaction mixture (i.e., following the reaction work-up). In one example, the process of reacting a compound of Formula 2 to form the bicyclic glycine-proline compound of Formula 1, as described herein, provides a bicyclic glycine-proline compound of Formula 1 following purification in at least 90%, at least 95%, at least 96%, at least 97%, at least 98%, at least 99%, at least 99.5%, at least 99.8%, least 99.9%, or 100% purity. In one example, the process of reacting a compound of Formula 2 to form the bicyclic glycine-proline compound of Formula 1, as described herein, provides a bicyclic glycine-proline compound of Formula 1 following recrystallization in at least 95%, at least 96%, at least 97%, at least 98%, at least 99%, at least 99.5%, at least 99.8%, at least 99.9%, or 100% purity. In one example, the process of reacting a compound of Formula 2 to form the bicyclic glycine-proline compound of Formula 1, as described herein, provides a compound of Formula 1 following column chromatography in at least 95%, at least 96%, at least 97%, at least 98%, at least 99%, at least 99.5%, at least 99.8%, at least 99.9%, or 100% purity.

In some embodiments, the process for preparing an amide compound of Formula 2 occurs with a starting material amount of a compound of Formula 4 of at least 1 g, at least 10 g, at least 50 g, at least 100 g, at least 500 g, at least 1 kg, at least 10 kg, at least 20 kg, at least 30 kg, or at least 40 kg. That is, the process for preparing an amide compound of Formula 2 occurs on at least 1 g, at least 10 g, at least 50 g, at least 100 g, at least 500 g, at least 1 kg, at least 10 kg, at least 20 kg, at least 30 kg, or at least 40 kg scale. In one example, the process for preparing a compound of Formula 2 occurs on at least 1 g, at least 10 g, at least 50 g, at least 100 g, at least 500 g, at least 1 kg, at least 10 kg, at least 20 kg, at least 30 kg, or at least 40 kg scale. In one example, the process for preparing an amide compound of Formula 2 occurs with a starting material amount of a compound of Formula 4 of at least 1 g, at least 10 g, at least 50 g, at least 100 g, at least 500 g, at least 1 kg, at least 10 kg, at least 20 kg, at least 30 kg, or at least 40 kg scale.

In some embodiments, the process provides a molar conversion of a compound of Formula 4 to a compound of Formula 2 of at least 50%, at least 60%, at least 70%, at least 80%, at least 90%, at least 95%, at least 98%, or at least 99%, as measured by HPLC. In some embodiments, the process of preparing an amide compound of Formula 2 provides a conversion of a compound of Formula 4 to a compound of Formula 2 of at least 50%, at least 60%, at least 70%, at least 80%, at least 90%, at least 95%, at least 98%, or at least 99%, as measured by HPLC.

In some embodiments, the process provides a yield of a compound of Formula 2 of at least 30%, at least 40%, at least 50%, at least 60%, at least 70%, or at least 80%, at least 90%, at least 95%, or at least 98%, as determined from a compound of Formula 4 or Formula 5 starting materials. That is, in some embodiments, the process of reacting a compound of Formula 4 with a compound of Formula 5 under amide coupling conditions, to form a compound of Formula 2, as described herein, provides at least 30%, at least 40%, at least 50%, at least 60%, at least 70%, or at least 80%, at least 90%, at least 95%, or at least 98% yield of a compound of Formula 2. In some embodiments, the process of reacting a compound of Formula 4 with a compound of Formula 5 under amide coupling conditions, to form a compound of Formula 2, as described herein, provides between about 30% and 100%, between about 50% and 99%, or between about 70% and 99% yield of a compound of Formula 2.

In some embodiments, the process described herein provides a compound of Formula 2 in high purity. In some embodiments, the process of reacting a compound of Formula 4 with a compound of Formula 5 under amide coupling conditions, to form a compound of Formula 2, as described herein, provides a compound of Formula 2 in at least 80%, at least 85%, at least 90%, at least 95%, at least 96%, at least 97%, at least 98%, at least 99%, at least 99.5%, at least 99.8%, at least 99.9%, or 100% purity. In one example, the process of reacting a compound of Formula 4 with a compound of Formula 5 under amide coupling conditions, to form a compound of Formula 2, as described herein, provides a compound of Formula 2 in at least 80%, at least 85%, at least 90%, at least 95%, at least 96%, at least 97%, at least 98%, at least 99%, at least 99.5%, at least 99.8%, at least 99.9%, or 100% purity of the product in the crude reaction mixture. In one example, the process of reacting a compound of Formula 4 with a compound of Formula 5 under amide coupling conditions, to form a compound of Formula 2, as described herein, provides a compound of Formula 2 in at least 80%, at least 85%, at least 90%, at least 95%, at least 96%, at least 97%, at least 98%, at least 99%, or 100% purity of the product isolated from the reaction mixture (i.e., following the reaction work-up). In one example, the process of reacting a compound of Formula 4 with a compound of Formula 5 under amide coupling conditions, to form a compound of Formula 2, as described herein, provides a compound of Formula 2 in at least 90%, at least 95%, at least 96%, at least 97%, at least 98%, at least 99%, at least 99.5%, at least 99.8%, at least 99.9%, or 100% purity following purification. In one example, the process of reacting a compound of Formula 4 with a compound of Formula 5 under amide coupling conditions, to form a compound of Formula 2, as described herein, provides a compound of Formula 2 in at least 90%, at least 95%, at least 96%, at least 97%, at least 98%, at least 99%, at least 99.5%, at least 99.8%, at least 99.9%, or 100% purity following recrystallization. In one example, the process of reacting a compound of Formula 4 with a compound of Formula 5 under amide coupling conditions, to form a compound of Formula 2, as described herein, provides a compound of Formula 2 in at least 95%, at least 96%, at least 97%, at least 98%, at least 99%, or 100% purity following column chromatography.

In some embodiments, the process for preparing a compound of Formula 4 occurs with a starting material amount of a compound of Formula 6 of at least 1 g, at least 10 g, at least 50 g, at least 100 g, at least 500 g, at least 1 kg, at least 10 kg, at least 25 kg, or at least 50 kg. That is, the process for preparing a compound of Formula 4 occurs on at least 1 g, at least 10 g, at least 50 g, at least 100 g, at least 500 g, at least 1 kg, at least 10 kg, at least 25 kg, or at least 50 kg scale. In one example, the process for preparing a compound of Formula 4 occurs with a starting material amount of a compound of Formula 6 of at least 1 g, at least 10 g, at least 50 g, at least 100 g, at least 500 g, at least 1 kg, at least 10 kg, at least 25 kg, or at least 50 kg scale.

In some embodiments, the process provides a molar conversion of a compound of Formula 6 to a compound of Formula 4 of at least 40%, at least 50%, at least 60%, at least 70%, at least 75%, at least 80%, at least 90%, or at least 95%, as measured by HPLC. In some embodiments, the process of reacting a compound of Formula 6 under acid-catalysed esterification conditions to form the compound of Formula 4, provides a conversion of a compound of Formula 6 to a compound of Formula 4 of at least 40%, at least 50%, at least 60%, at least 70%, at least 75%, at least 80%, at least 90%, or at least 95% as measured by HPLC.

In some embodiments, the process provides a yield of a compound of Formula 4 of at least 20%, at least 30%, at least 40%, at least 50%, at least 60%, at least 70%, at least 80%, at least 90%, or at least 95% as determined from a compound of Formula 6 starting material. That is, in some embodiments, the process of reacting a compound of Formula 6 under acid-catalysed esterification conditions, to form a compound of Formula 4, as described herein, provides at least 20%, at least 30%, at least 40%, at least 50%, at least 60%, at least 70%, at least 80%, at least 90%, or at least 95% yield of a compound of Formula 4. In some embodiments, the process of reacting a compound of Formula 6 under acid-catalysed esterification conditions, to form a compound of Formula 4, as described herein, provides between about 30% and 100%, between about 50% and 99%, or between about 70% and 98% yield of a compound of Formula 4.

In some embodiments, the process described herein provides a compound of Formula 4 in high purity. In some embodiments, the process of reacting a compound of Formula 6 under acid-catalysed esterification conditions, to form a compound of Formula 4, as described herein, provides a compound of Formula 4 in at least 80%, at least 85%, at least 90%, at least 95%, at least 96%, at least 97%, at least 98%, at least 99%, at least 99.5%, at least 99.8%, at least 99.9%, or 100% purity. In one example, the process of reacting a compound of Formula 6 under acid-catalysed esterification conditions, to form a compound of Formula 4, as described herein, provides a compound of Formula 4 in at least 80%, at least 85%, at least 90%, at least 95%, at least 96%, at least 97%, at least 98%, at least 99%, at least 99.5%, at least 99.8%, or at least 99.9% purity of the product in the crude reaction mixture. In one example, the process of reacting a compound of Formula 6 under acid-catalysed esterification conditions, to form a compound of Formula 4, a described herein, provides a compound of Formula 4 in at least 80%, at least 85%, at least 90%, at least 95%, at least 96%, at least 97%, at least 98%, at least 99%, at least 99.5%, at least 99.8%, or at least 99.9% purity of the product isolated from the reaction mixture (i.e., following the reaction work-up). In one example, the process of reacting a compound of Formula 6 under acid-catalysed esterification conditions, to form a compound of Formula 4, as described herein, provides a compound of Formula 4 in at least 90%, at least 95%, at least 96%, at least 97%, at least 98%, at least 99%, at least 99.5%, at least 99.8%, at least 99.9%, or 100% purity following purification. In one example, the process of reacting a compound of Formula 6 under acid-catalysed esterification conditions, to form a compound of Formula 4, as described herein, provides a compound of Formula 4 in at least 95%, at least 96%, at least 97%, at least 98%, at least 99%, at least 99.5%, at least 99.8%, at least 99.9%, or 100% purity following recrystallization. In one example, the process of reacting a compound of Formula 6 under acid-catalysed esterification conditions, to form a compound of Formula 4, as described herein, provides a compound of Formula 4 in at least 95%, at least 96%, at least 97%, at least 98%, at least 99%, at least 99.5%, at least 99.8%, at least 99.9%, or 100% purity following column chromatography.

In some embodiments, the overall process of the synthesis of a bicyclic glycine-proline compound of Formula 1 from a compound of Formula 6 (i.e., conversion of Formula 6 to Formula 4 to Formula 2 to Formula 1) provides an overall yield of between about 20% and about 95%, between about 30% and about 90%, between about 40% and about 85%, or between about 60% and about 80%. In one example, the process for preparing a bicyclic glycine-proline compound of Formula 1 from a compound of Formula 6 provides an overall yield of at least about 20%, 25%, 30%, 35%, 40%, 45%, 50%, 60%, 70%, 80%, 90%, or 95%.

### Compounds

In some embodiments, there is provided a bicyclic glycine-proline compound of Formula 1:

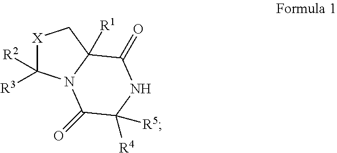

wherein X, R¹, R², R³, R⁴, and R⁵ are each as described herein, prepared by any one or more of the processes as described herein.

In some embodiments, there is provided a bicyclic glycine-proline compound of Formula 1a:

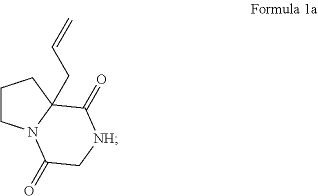

prepared by any one or more of the processes as described herein.

In some embodiments, there is provided a bicyclic glycine-proline compound of Formula 1b:

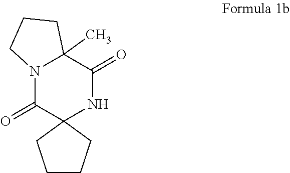 [SMILES: CC12CCCN1C(=O)C1(CCCC1)NC2=O]

prepared by any one or more of the processes as described herein.

In some embodiments, there is provided a bicyclic glycine-proline compound of Formula 1c:

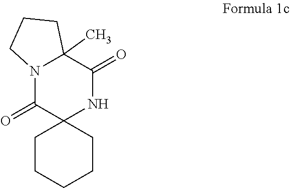 [SMILES: CC12CCCN1C(=O)C1(CCCCC1)NC2=O]

prepared by any one or more of the processes as described herein.

In some embodiments, there is provided a bicyclic glycine-proline compound of Formula 1a(R):

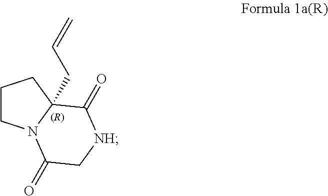

prepared by any one or more of the processes as described herein.

In some embodiments, there is provided a bicyclic glycine-proline compound of Formula 1a(S):

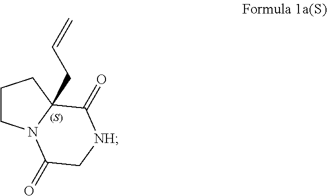

prepared by any one or more of the processes as described herein.

In some embodiments, there is provided a compound of Formula 2:

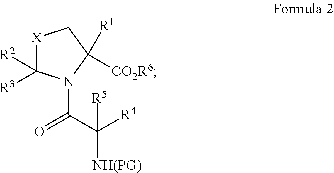

wherein X, R¹, R², R³, R⁴, R⁵, R⁶, and PG are as described herein, prepared by any one or more of the processes as described herein.

In some embodiments, there is provided a compound of Formula 2a:

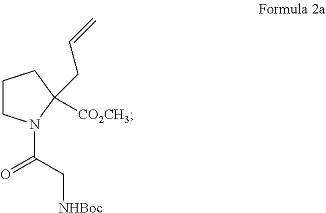 [SMILES: C=CCC1(C(=O)OC)CCCN1C(=O)CNC(=O)OC(C)(C)C]

prepared by any one or more of the processes as described herein.

In some embodiments, there is provided a compound of Formula 2a(R):

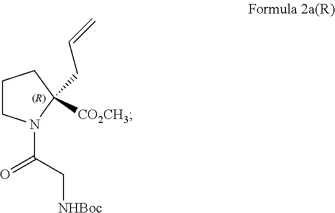

prepared by any one or more of the processes as described herein.

In some embodiments, there is provided a compound of Formula 2a(S):

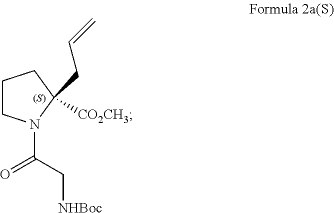

prepared by any one or more of the processes as described herein.

In some embodiments, there is provided a compound of Formula 3:

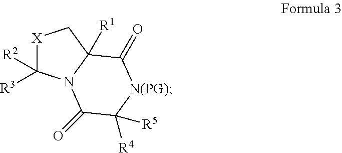

wherein X, R¹, R², R³, R⁴, R⁵, and PG are as described herein, prepared by any one or more of the processes as described herein.

In some embodiments, there is provided a compound of Formula 3a:

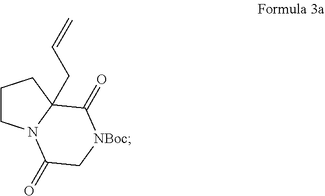 [SMILES: C=CCC12CCCN1C(=O)CN(C(=O)OC(C)(C)C)C2=O]

prepared by any one or more of the processes as described herein.

In some embodiments, there is provided a compound of Formula 3a(R):

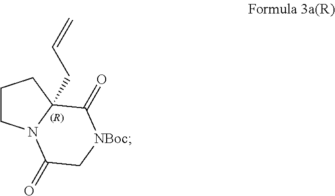

prepared by any one or more of the processes as described herein.

In some embodiments, there is provided a compound of Formula 3a(S):

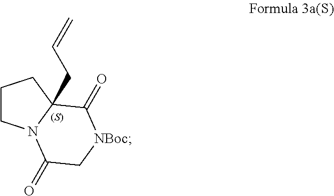

prepared by any one or more of the processes as described herein.

In some embodiments, there is provided a compound of Formula 4 or salt thereof:

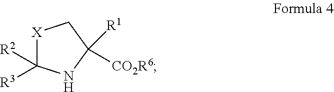

wherein X, R¹, R², R³, and R⁶ are as described herein, prepared by any one or more of the processes as described herein.

In some embodiments, there is provided a compound of Formula 4a or salt thereof:

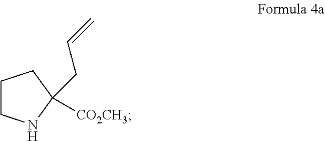

prepared by any one or more of the processes as described herein.

In some embodiments, there is provided a compound of Formula 4a(R) or salt thereof:

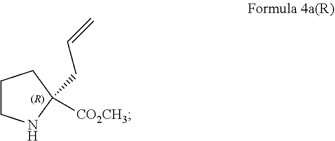

prepared by any one or more of the processes as described herein.

In some embodiments, there is provided a compound of Formula 4a(S):

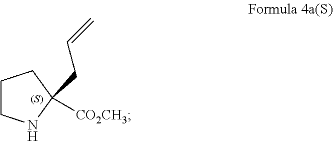

prepared by any one or more of the processes as described herein.

### Compositions

Whilst a compound of Formula 1 or salt thereof may in some embodiments be administered alone, it is more typically administered as part of a pharmaceutical composition or formulation. Thus, the present disclosure also provides a pharmaceutical composition comprising a compound of Formula 1 or salt thereof and a pharmaceutically acceptable excipient. The pharmaceutical composition comprises one or more pharmaceutically acceptable diluents, carriers, or excipients (collectively referred to herein as “excipient” materials).

The present disclosure also provides pharmaceutical formulations or compositions, both for veterinary and for human medical use, which comprise compounds of Formula 1 of the present disclosure or a pharmaceutically acceptable salt thereof, with one or more pharmaceutically acceptable carriers, and optionally any other therapeutic ingredients, stabilisers, or the like. The carrier(s) must be pharmaceutically acceptable in the sense of being compatible with the other ingredients of the formulation and not unduly deleterious to the recipient thereof.

The pharmaceutical formulations may conveniently be presented in unit dosage form and may be prepared by any of the methods well known in the art of pharmacy. All methods include the step of bringing a compound of Formula 1 or salt thereof into association with the excipient that constitutes one or more necessary ingredients. In general, the formulations are prepared by uniformly and intimately bringing into association the active ingredient with liquid carriers or finely divided solid carriers or both, and then, if necessary, shaping the product into the desired formulation.

A tablet may be made for example by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active, or dispersing agent. Moulded tablets may be made by moulding in a suitable machine a mixture of the powdered compound moistened with an inert liquid diluent. The tablets may be optionally coated or scored, and may be formulated so as to provide slow, pulsated or controlled release of the compound of Formula 1. The compound of Formula 1 can, for example, be administered in a form suitable for immediate release or controlled release. Immediate release or controlled release can be achieved by the use of suitable pharmaceutical compositions comprising a compound of Formula 1 or, particularly in the case of controlled release, by the use of devices such as subcutaneous implants or osmotic pumps. A compound of Formula 1 may also be administered liposomally.

In some embodiments, there is provided a composition comprising a bicyclic glycine-proline compound of Formula 1a and one or more excipients according to any embodiments or examples thereof as described herein:

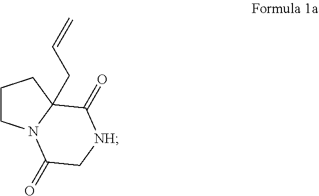

wherein any impurities, if present, are in an amount (weight % of the amount of the compound of Formula 1a) of less than about 5, 4, 3, 2, 1, 0.5, 0.1, 0.05, 0.01, 0.005, 0.001, 0.0005 or 0.0001. The composition may be substantially free of any impurities. The impurities may be selected from any one or more of the by-products or reagents used in the processes as described herein. The composition may be a pharmaceutical composition comprising one or more pharmaceutically acceptable excipients according to any embodiments or examples thereof as described herein.

In some embodiments, there is provided a composition comprising a bicyclic glycine-proline compound of Formula 1a(R) and one or more excipients according to any embodiments or examples thereof as described herein:

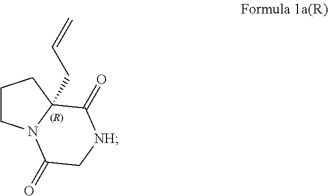

wherein any impurities, if present, are in an amount (weight % of the amount of the compound of Formula 1a(R)) of less than about 5, 4, 3, 2, 1, 0.5, 0.1, 0.05, 0.01, 0.005, 0.001, 0.0005 or 0.0001. The composition may be substantially free of any impurities. The impurities may be selected from any one or more of the by-products or reagents used in the processes as described herein. The composition may be a pharmaceutical composition comprising one or more pharmaceutically acceptable excipients according to any embodiments or examples thereof as described herein.

In some embodiments, there is provided a composition comprising a bicyclic glycine-proline compound of Formula 1a(S) and one or more excipients according to any embodiments or examples thereof as described herein:

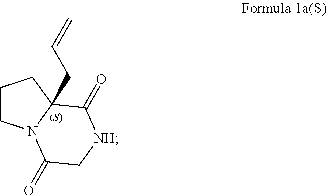

wherein any impurities, if present, are in an amount (weight % of the amount of the compound of Formula 1a(S)) of less than about 5, 4, 3, 2, 1, 0.5, 0.1, 0.05, 0.01, 0.005, 0.001, 0.0005 or 0.0001. The composition may be substantially free of any impurities. The impurities may be selected from any one or more of the by-products or reagents used in the processes as described herein. The composition may be a pharmaceutical composition comprising one or more pharmaceutically acceptable excipients according to any embodiments or examples thereof as described herein.

The present disclosure will now be described with reference to the following examples which illustrate some particular aspects of the present disclosure. However, it is to be understood that the particularity of the following description of the present disclosure is not to supersede the generality of the preceding description of the present disclosure.

### Examples

The present disclosure is further illustrated by the following examples. These examples are offered by way of illustration only and are not intended to limit the scope of the disclosure.

### Abbreviations

API Active pharmaceutical ingredient
AG Activating group
Alloc Aallyloxycarbonyl protecting group
Aq. Aqueous
BOP Benzotriazol-1-yloxy)tris(dimethylamino)phosphonium hexafluorophosphate
BOP—Cl Bis(2-oxo-3-oxazolidinyl)phosphinic chloride
Boc tert-Butyloxycarbonyl protecting group
Brine Saturated aqueous sodium chloride solution
nBuLi n-Butyl lithium
Cbz Benzyl carbamate or carboxybenzyl protecting group
CDI N,N′-Carbonyldiimidazole
COMU (1-Cyano-2-ethoxy-2-oxoethylidenaminooxy)dimethylamino-morpholino-carbenium hexafluorophosphate
CPME Cyclopentyl methyl ether
DIPEA N,N-Diisopropylethylamine
DBU 1,8-Diazabicyclo[5.4.0]undec-7-ene
DCC Dicyclohexylcarbodiimide
DCM Dichloromethane
DIC Diisopropylcarbodiimide
Dioxane 1,4-Dioxane
DIPEA N,N-Diisopropylethylamine
DMAP Dimethylaminopyridine
DMC Dimethyl carbonate
DME Dimethoxyethane or Monoglyme
DMF N,N-Dimethylformamide
DMPU N,N-Dimethylpropyleneurea
DMSO Dimethylsulfoxide
DppCl Diphenylphosphinic acid chloride
EDC 1-Ethyl-3-(3-dimethylaminopropyl)carbodiimide
EDC·HCl 1-Ethyl-3-(3-dimethylaminopropyl)carbodiimide hydrochloride
Eq. Equivalents
EtOAc Ethyl acetate
EtOH Ethanol
Et₃N Triethylamine
Fmoc 9-Fluorenylmethyloxycarbonyl
Gly Glycine; Gly-OH
HATU Hexafluorophosphate Azabenzotriazole Tetramethyl Uronium
HBTU Benzotriazole-N,N,N′,N′-tetramethylurea hexafluorophosphate
HCl Hydrogen chloride
HCTU O-(6-Chlorobenzotriazol-1-yl)-N,N,N′,N′-tetramethyluronium hexafluorophosphate
HDPE High Density Polyethylene
HOBt 1-Hydroxybenzotrizole
HOPO 2-Hydroxypyridine-N-oxide
HPLC High Performance Liquid Chromatography
IBCF Isobutyl chloroformate
IPA Isopropyl alcohol or 2-propanol or iPrOH
iPAC Isopropyl acetate
K₂CO₃ Potassium carbonate
KHMDS Potassium hexamethyldisilazide or Potassium bis(trimethylsilyl)amide
KOEt Potassium ethoxide
KOH Potassium hydroxide
KOME potassium methoxide
KOtBu Potassium tert-butoxide
LC Liquid chromatography
LDA Lithium diisopropylamide
LDEA Lithium diethylamide
LiHMDS Lithium hexamethyldisilazide
MBK Methylbutyl ketone
MeCN Acetonitrile
MEK Methylethyl ketone
MeOAc Methyl acetate
MeOH Methanol
MeOZ p-Methoxybenzyl carbonyl protecting group
MeTHF Methyltetrahydrofuran
MIBK Methylisobutyl ketone
MIPK Methylisopropyl ketone
MSA Methane sulphonic acid
MTBE tert-Butyl methyl ether
NaH Sodium hydride
NaHCO₃Sodium bicarbonate
NaHMDS Sodium hexamethyldisilazide or Sodium bis(trimethylsilyl)amide
NaNH₂ Sodamide
NaOEt Sodium ethoxide
NaOH Sodium hydroxide
NaOMe Sodium methoxide
NaOtBu Sodium tert-butoxide
NaNH₂ Sodamide
Na₂SO₄ Sodium sulfate
NMM N-Methylmorpholine
NMP N-Methyl-2-pyrrolidone
NMR Nuclear Magnetic Resonance spectrometry
PC Propylene carbonate
PG Protecting group
PivCl Pivaloyl chloride
PyAOP 7-Azabenzotriazol-1-yloxy)tripyrrolidinophosphonium hexafluorophosphate
PyBOP 1H-benzotriazol-1-yloxytripyrrolidinyl hexafluorophosphate
PyBrOP Bromotripyrrolidinophosphonium hexafluorophosphate
qNMR Quantitative Nuclear Magnetic Resonance spectrometry
RT Room temperature
SOCl₂ Thionyl chloride
Su Succinimide
TATU O-(7-Azabenzotriazol-1-yl)-N,N,N′,N′-tetramethyluronium tetrafluoroborate
TBTU 2-(1H-benzotriazo L-1-yl)-1,1,3,3-Tetramethylurea tetrafluoroborate
t-BuOH t-Butanol
TCT Cyanuric chloride
THF Tetrahydrofuran
TLC Thin layer chromatography
TFA Trifluoroacetic acid or Trifluoroacetyl protecting group
TMEDA Tetramethylethylenediamine
Tos p-Toluensulfonyl or p-Tosyl protecting group or para-tosyl
Trt Trityl protecting group
TTIP Titanium tetraisopropoxide or Titanium isopropoxide
T₃P Propyl phosphonic anhydride

### Overall Synthesis

A non-limiting illustration of the overall synthesis used to obtain the final target compound 1 (NNZ-2591) with respect to the below Examples and compounds of Formulae 1 to 6 is provided as follows:

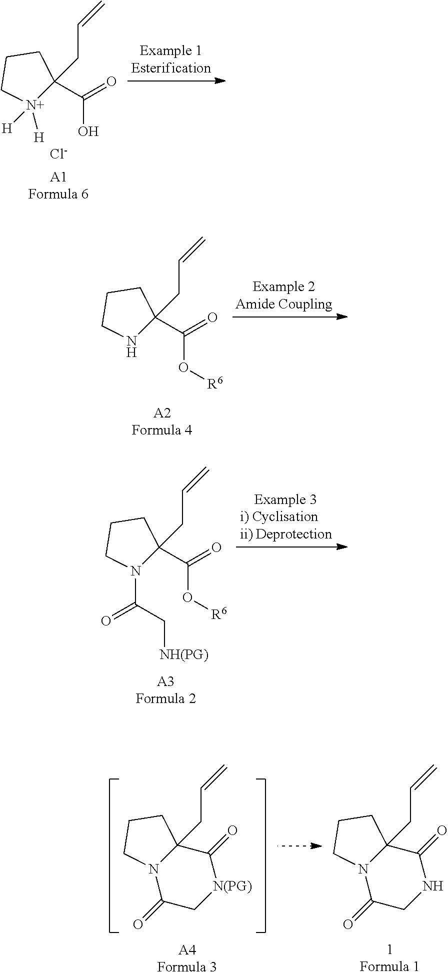

### Example 1: Synthesis of Compound A2

### Example 1a: Synthesis of Compound A2 Using Methanesulfonic Acid

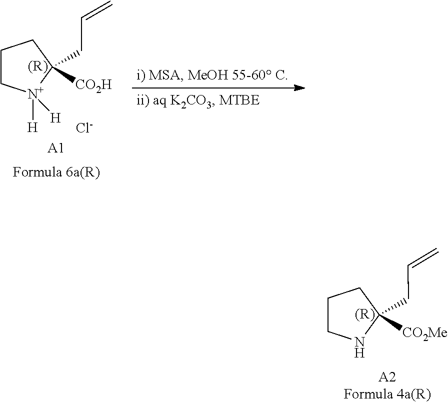

To a 1000 L vessel with an inert atmosphere via a vacuum-nitrogen purge cycle, A1 [49.8 kg, 259.8 moles, 1.0 eq.] was charged, followed by methanol [199 kg L, 251 L, 5.0 vol.] and the resultant mixture was stirred at 20° C. for 15 minutes to dissolve the solid. The vessel was then charged with methanesulfonic acid (50.4 kg, 524 moles, 2.0 eq) over 30 minutes, maintaining the batch temperature below 35° C. The batch was then aged at about 58° C. for 67 hours. The reaction was monitored by HPLC; after 67 hours, by LC area %, 0.6% of A1 remained with respect to A2.

The reaction mixture was cooled to 20° C. and then concentrated under reduced pressure from about 330 L to 105 L, maintaining the temperature of the vessel contents below 50° C.

The batch was cooled to 15° C. and the vessel was charged with water [50 kg, 50 L; 1.0 vol.] over 10 minutes, maintaining the batch temperature at ≤20° C. The vessel was then charged with MTBE [222 kg, 300 L, 6.0 vol.], followed by 24 wt % aqueous potassium carbonate (made in a separate 400 L vessel by the dissolution of potassium carbonate [72.1 kg, 522 moles] in water [225 kg, 225 L]) which was charged over 2 hours, maintaining the batch temperature at ≤20° C., followed by a line rinse with water (25 L, 0.5 vol.).

After warming to 22° C., the aqueous phase was separated and extracted with MTBE [207 kg, 280 L, 5.6 vol] and the resultant aqueous phase was separated and again extracted with MTBE [222 kg, 300 L, 6.0 vol.]. The MTBE phases were combined and at 22° C. were washed with water [100 kg, 100 L, 2.0 vol].

The resultant organic phase was concentrated under reduced pressure, maintaining the batch temperature at ≤50° C. The batch was cooled to 20° C. and the vessel was charged with MTBE [74.1 kg, 100 L, 2.0 vol.]. The batch was then concentrated under reduced pressure from approximately 200 L to 95 L, maintaining the batch temperature at <50° C. The batch was cooled to 20° C. and sampled for analysis of water and methanol content. The water content was determined by Karl-Fisher titration to be 0.45 wt %. The methanol content was determined by ¹H NMR to be 0.35 wt %. The solution was transferred to a HDPE-lined drum to give A2 as an MTBE solution (79.1 kg); the wt % of A2 in solution was determined by HPLC. Total weight of solution was 79.1 kg and the wt % of A2 in solution was 51.1 wt %. Yield of pure A2 is therefore 40.4 kg (91.9%).

### Example 1b: Synthesis of Compound A2 Using Acetyl Chloride

Adding acetyl chloride (4 equiv), over 1 hour, to a solution of A1 in methanol (5 vol) at 0° C. and then heating to 50-55° C. for 18 hours resulted in 88% conversion of A1 to desired ester A2 (by HPLC). Therefore, a further 2 equivalents of acetyl chloride was added (after cooling the reaction mixture back to 0° C.) and the reaction was aged at 50-55° C. for a further 6 hours. This drove conversion to 92%. After cooling the final reaction mixture to room temperature and concentrating to 2 volumes, the solution was azeotropically dried to 1.5% water by distilling with toluene (2×5 vol) and subsequently with IPA (2×10 vol). Crystallization and isolation from 4:1 MTBE/IPA (10 vol) provided the HCl salt of A2 at 65% yield.

### Example 1c: Synthesis of Compound A2 Using Trimethylsilyl Chloride

Adding trimethylsilyl chloride (6 equiv), over 15 minutes, to a solution of A1 in methanol (10 vol) at 0° C. and then heating to 45-50° C. for 20 hours resulted in 91% conversion of A1 to desired ester A2 (by HPLC). Therefore, a further 1 equivalent of trimethylsilyl chloride was added (after cooling the reaction mixture to 5° C.) and the reaction was aged at 45-50° C. for a further 20 hours. This drove conversion to 97%. The final reaction mixture was biphasic at this point. The upper layer was discarded and the lower methanol layer was concentrated to 5 volumes before azeotropically drying (4×10 vol IPA) and isolating from 4:1 MTBE/IPA (10 vol). This afforded the desired A2 HCl salt in 79% yield.

### Example 1d: Synthesis of Compound A2 Using Thionyl Chloride

Adding a solution of A1 in methanol (2 vol) to 4 equiv of thionyl chloride in methanol (5 vol; prepared by slow addition of SOCl₂ to methanol at <25° C.) at 5° C. and then heating to 50-55° C. for 20 hours resulted in 95% conversion of A1 to desired ester A2 (by HPLC). Therefore, a further 1 equivalent of thionyl chloride was added (after cooling the reaction mixture back to 5° C.) and the reaction was aged at 50-55° C. for a further 6 hours. This drove conversion to 97%. After cooling the final reaction mixture to room temperature and concentrating to 2 volumes, the solution was diluted with toluene (5 vol) and then re-concentrated to 2 volumes. Distilling several times with IPA (3×5 vol) was then carried out to azeotropically dry the solution and afford a thick suspension of A2 HCl salt in 2 volumes of IPA. Heating the final slurry to 60-70° C. re-dissolved the solid, and A2 HCl salt was then recrystallized by cooling to room temperature, with seeding at 35° C. MTBE (8 vol) was then added to improve recovery, and the HCl salt of A2 was then isolated, by filtration, in 85% yield.

### Example 1e: Synthesis of Compound A2 Using Oxalyl Chloride

Adding a solution of 1 equiv of oxalyl chloride with 5 mol % DMF in dichloromethane to a solution of A1 in dichloromethane, followed by reaction with 1.5 equiv of methanol, resulted in 99% conversion to A2 after 1 hour. The solution was concentrated to dryness, affording the A2 HCl salt at a yield of 90%.

### Example 2: Synthesis of Compound A3

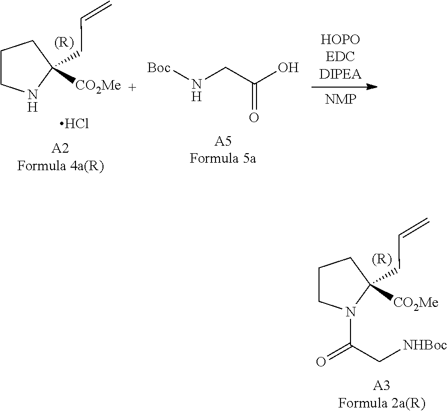

A 1000 L vessel was charged with HOPO [7.99 kg, 71.5 moles, 0.30 eq.], EDC hydrochloride [59.4 kg, 310 moles, 1.30 eq.] and then put under an inert atmosphere via a vacuum-nitrogen purge cycle, before being charged with NMP [122 kg, 118 L, 3.0 vol.]. The vessel was then charged with a solution of A2 in MTBE [51.1 wt %, 78.8 kg, 40.3 kg pure A2, 238 moles, 1.0 eq.], an MTBE line wash [18.5 kg, 13.7 L, 0.30 vol.], and DIPEA [46.2 kg, 357 moles, 1.50 eq.].

A separate 400 L vessel was charged with A5 (Boc-Gly-OH) [45.9 kg, 262 moles, 1.1 eq.] and NMP [104 kg, 101 L, 2.5 vol.] and the resultant mixture was transferred from the 400 L vessel to the 1000 L vessel over 32 minutes, maintaining the batch temperature at ≤25° C. The 400 L vessel was charged with a line wash of NMP [15.4 kg, 15.0 L, 0.4 vol.] which was then transferred to the 1000 L vessel. The resultant mixture was aged at 20° C. for 15 hours.

Analysis of a sample of the reaction mixture by HPLC indicated that the conversion was effectively 100% as the amount of A2 present was below the limit of detection.

The vessel was charged with MTBE [179 kg, 242 L, 6 vol.] and then, maintaining the batch temperature below 25° C., a 10% w/w citric acid solution [7.0 vol.], made in a separate 400 L vessel by dissolving citric acid [28.3 kg] in purified water [254 kg], was charged, over 43 minutes, followed by a line rinse with purified water [22.4 kg, 0.56 vol.]. The phases were separated and the aqueous phase extracted with further MTBE [212 kg, 287 L, 7 vol.]. The combined organic phases were washed with a 5% w/w sodium bicarbonate solution [10 vol.], made in a separate 400 L vessel by dissolving sodium bicarbonate [20.2 kg] in water [383 kg+25 kg line rinse], followed by an additional wash with water [202 kg, 5.0 vol.] The resultant organic phase was then concentrated under reduced pressure, keeping the batch temperature below 40° C., from approximately 600 L to approximately 170 L.

The batch was cooled and at 20° C. the solution was seeded with A3 [17.0 g], with crystallization subsequently evident. The resultant suspension was aged at 20° C., with stirring, for 16 hours. n-Heptane [331 kg, 484 L, 12 vol.] was charged to the vessel over 90 minutes and the resultant suspension was aged at 20° C. for a further 1 hour. The suspension was then concentrated under reduced pressure from approximately 640 L to approximately 240 L, maintaining the batch temperature at ≤40° C. The batch was cooled to 20° C. and then aged at 20° C. for 18 hours. The vessel was rinsed with n-heptane [82.7 kg, 121 L, 3 vol.] and the rinse was used to wash the filter cake. The filter cake was then transferred on to trays and dried in a tray dryer at 30° C. and under vacuum with a nitrogen sweep.

The solid was transferred to into a double-bagged polythene-lined HDPE keg to give A3 as a white crystalline solid [69.9 kg, 98.7 wt % purity by HPLC; 88.8% corrected yield].

### Example 3: Synthesis of Compound 1

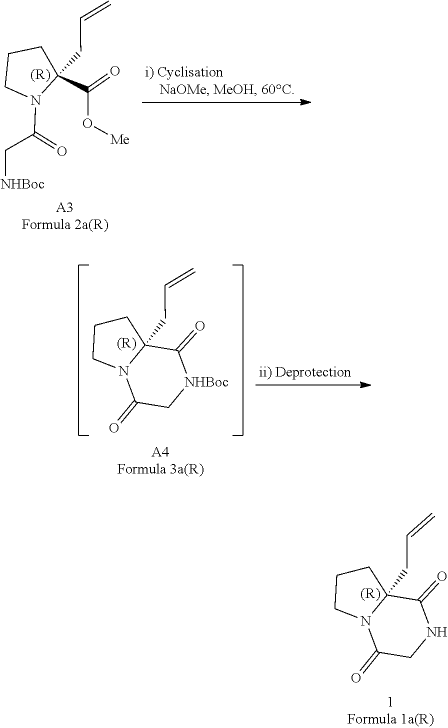

A 1000 L vessel was charged with A3 [105 kg, 322 moles, 1.0 eq.] and then put under an inert atmosphere, before adding methanol [415 kg, 524 L, 5.0 vol.]. The vessel was then charged with a solution of sodium methoxide in methanol [5.4 M, 30 wt %, 29.0 kg, 8.69 kg wt % corrected; 161 moles, 0.5 eq.] over 47 minutes, ensuring a batch temperature of <25° C., followed by further methanol [3.1 kg, 3.9 L] as a line rinse. The one pot cyclisation reaction forms the cyclised A4 intermediate in situ while also deprotecting the A4 intermediate to form the final target compound 1. The resultant mixture was heated to 59° C. and aged, with stirring, at 59° C. for 15 hours, to form compound 1 (in situ via the A4 intermediate). Analysis of a sample of the reaction mixture by HPLC indicated that the quantity of A3 remaining was 0.1% by area.

To quench and neutralize the base in the reaction, the mixture was cooled to 16° C. and the vessel was charged with 6 N HCl in isopropyl alcohol [24.3 kg, 167 moles, 0.52 eq.] over 23 minutes, ensuring a batch temperature of <25° C., followed by methanol [3.1 kg, 3.9 L] as a line rinse. The resultant mixture was concentrated under reduced pressure from approximately 680 L to approximately 158 L, maintaining the batch temperature at ≤40° C. The batch was cooled to 21° C. and charged with isopropyl acetate [595 kg, 684 L] and the resultant mixture was aged, with stirring, at 20° C. for 17 hours and then sampled to determine the ratio of methanol to isopropyl acetate, which was 28.4 mol % methanol with respect to isopropyl acetate by ¹H NMR.

The resultant mixture was filtered and the filtrate collected in drums. The vessel was charged with isopropyl acetate [82.4 kg, 94.8 L, 0.9 vol.] and methanol [8.6 kg, 10.9 L, 0.1 vol.], which were used to wash the vessel. The mixture was discharged from the vessel, passed through the filter and this wash filtrate was collected in a separate drum.

The solutions were weighed and the samples were analysed by HPLC (Initial Filtrate: 8.52 wt % 1, total weight of solution: 685.4 kg; Wash Filtrate: 4.17 wt % 1, total weight of solution: 116.4 kg).

The solutions were combined and underwent a series of concentration steps under reduced pressure, including seeding with compound 1 and aging of the batch at 20° C. for 16 hours. Further concentration was followed by aging at 20° C. for 18 hours in approximately 50:50 n-heptane to isopropyl acetate. The contents were filtered and the filter cake dried at 41° C. under vacuum with a nitrogen sweep. The solid was transferred into double-bagged polythene-lined HDPE kegs to give compound 1 (NNZ-2591) as an off white to light orange crystalline solid [59.1 kg, 98.7 wt % purity (by qNMR); 93% corrected yield]. The product was further analyzed to provide 100% ee with total impurities by HPLC of about 0.06%.

### Example 4: Synthesis of Compound 1

A small-scale reaction adjusted for scale and following the synthesis described in Example 3 was performed using sodium bis(trimethylsilyl)amide (NaHMDS) as the base and tetrahydrofuran (THF) as the solvent at room temperature for 2 hours to afford compound 1 (NNZ-2591). Conversion from A3 to compound 1 was 95%.

### Example 5: Synthesis of Compound 1

A small-scale reaction adjusted for scale and following the synthesis described in Example 3 was performed using potassium butoxide (KOtBu) as the base and tetrahydrofuran (THF) as the solvent commenced at 0° C. and was allowed to warm to room temperature over 3 hours to afford compound 1 (NNZ-2591). Conversion from A3 to compound 1 was 99%.

### Example 6: Preparation of Ester Derivatives of Formula 4

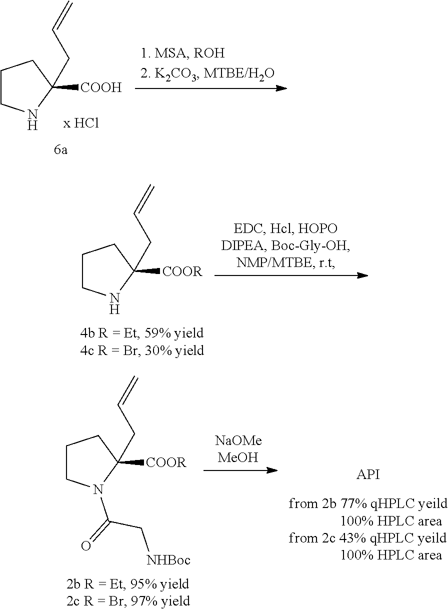

(R)-2-allylpyrrolidine-2-carboxylic acid hydrochloride 6a (A1; 1.0 g, 5.22 mmol, 1 eq) was placed in a vial and the specified alcohol (ethanol or benzyl alcohol) was added at ambient temperature. The solution was stirred for 1 minute (starting material completely dissolved, pale brown solution) and then the flask was placed in an ice bath. Methanesulfonic acid (0.678 mL, 10.44 mmol, 2 eq) was added dropwise via syringe under ice bath cooling. The vial was sealed, placed in a heating block and heated to 60° C. for 72 hours. A similar workup procedure to that described in Example 1a was employed.

The ethyl ester 4b was formed and easily purified by work up with a yield of 59%. This was converted to 2b with high yields (96%). This was then converted to the API (1, NNZ-2591) to give a qHPLC yield of 77%, with 100% HPLC area.

The purification of the benzyl ester 4c was more challenging. Compound 4c was obtained after the standard conditions, but there were significant other impurities present, including benzyl alcohol. After workup, the material was purified by column chromatography. This yielded 4c with 30% purity. Compound 4c was converted to 2c using standard amide coupling conditions, giving 97% yield. This was then converted to the API (1a, NNZ-2591) with a 43% yield by qHPLC.

### Example 7: Solvent Screen for Amide Coupling

A systematic screen of solvents was carried out for the amide coupling.

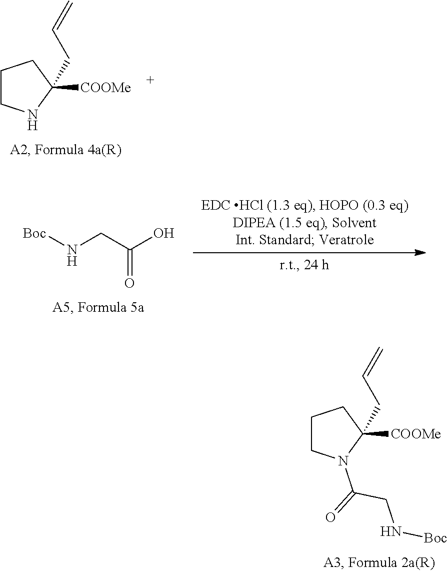 [SMILES: C=CC[C@@]1(C(=O)OC)CCCN1 ; CC(C)(C)OC(=O)NCC(=O)O ; C=CC[C@@]1(C(=O)OC)CCCN1C(=O)CNC(=O)OC(C)(C)C]

A vial was charged with (tert-butoxycarbonyl)glycine A5 (0.057 g, 0.325 mmol, 1.1 eq), EDC hydrochloride (0.074 g, 0.384 mmol, 1.3 eq), HOPO (9.9 mg, 0.089 mmol, 0.3 eq) and cooled with ice bath. Solvent (0.4 mL) and then DIPEA (0.077 mL, 0.443 mmol, 1.5 eq) were also added.

Veratrole (0.052 mL, 0.405 mmol, 1.37 eq) and methyl (R)-2-allylpyrrolidine-2-carboxylate (A2) (0.050 g, 0.295 mmol, 1 eq) were dissolved in solvent (0.4 mL). This solution was added to the glycine reaction mixture under ice bath cooling. Additional solvent (0.2 mL) was added and the resulting suspension was left to warm up to ambient temperature (22° C.) and stirred overnight.

After 20 hours, 10 μL of the reaction solution was dissolved in 1 mL MeCN and measured by HPLC. If this was not a solution, it was dissolved in 1 mL MeCN and 20 μL of the reaction mixture was dissolved in 1 mL MeCN.

TABLE 1
Solvents and reaction outcomes

| | | Yield of A3 by quantitative HPLC |
|---|---|---|
| Entry | Solvent | % |
| 1 | NMP/MTBE | 81 |
| 2 | NMP | 89 |
| 3 | MTBE | 53 |
| 4 | DMF | 60 |
| 5 | EtOAc | 59 |
| 6 | Pyridine | 86 |
| 7 | DCM | 33 |
| 8 | Acetone | 55 |
| 9 | MeCN | 33 |
| 10 | Toluene | 87 |
| 11 | IPA | 40 |
| 12 | MeTHF | 85 |
| 13 | DMSO | 60 |
| 14 | Dioxane | 81 |

### Example 8: Base Screen for Amide Coupling

A systematic screen of bases was carried out for the amide coupling.

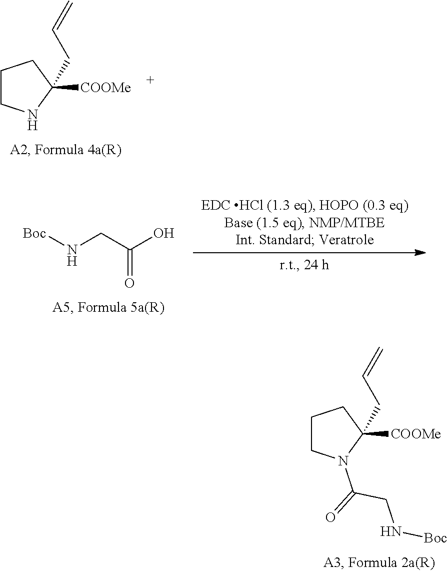 [SMILES: C=CC[C@@]1(C(=O)OC)CCCN1 ; CC(C)(C)OC(=O)NCC(=O)O ; C=CC[C@@]1(C(=O)OC)CCCN1C(=O)CNC(=O)OC(C)(C)C]

A vial was charged with (tert-butoxycarbonyl)glycine A5 (0.057 g, 0.325 mmol, 1.1 eq), EDC hydrochloride (0.074 g, 0.384 mmol, 1.3 eq), HOPO (9.9 mg, 0.089 mmol, 0.3 eq) and cooled with ice bath. NMP (0.4 mL) and then the specified base (0.443 mmol, 1.5 eq) were also added.

Veratrole (0.052 mL, 0.405 mmol, 1.37 eq) and methyl (R)-2-allylpyrrolidine-2-carboxylate (A2) (0.050 g, 0.295 mmol, 1 eq) were dissolved in NMP (0.4 mL). This solution was added to the glycine reaction mixture with ice bath cooling. MTBE (0.2 mL) was added and the resulting suspension was left to warm up to ambient temperature (22° C.) and stirred overnight. After 20 hours, 10 μL of the reaction solution was dissolved in 1 mL MeCN and measured by HPLC. If this was not a solution, it was dissolved in 1 mL MeOH and 20 μL of the reaction mixture was dissolved in 1 mL MeCN.

TABLE 2
Bases and reaction outcomes

| | | Yield of A3 by quantitative |
|---|---|---|
| Entry | Base | HPLC % |
| 1 | DIPEA | 84 |
| 2 | Et₃N | 77 |
| 3 | NaHCO₃ | 76 |
| 4 | KOtBu | 76 |
| 5 | Pyridine | 81 |
| 6 | Lutidine | 84 |
| 7 | NMM | 88 |
| 8 | NaOMe | 19 |

### Example 9: Amide Reagent for Amide Coupling

A systematic screen of coupling reagents was carried out for the amide coupling.

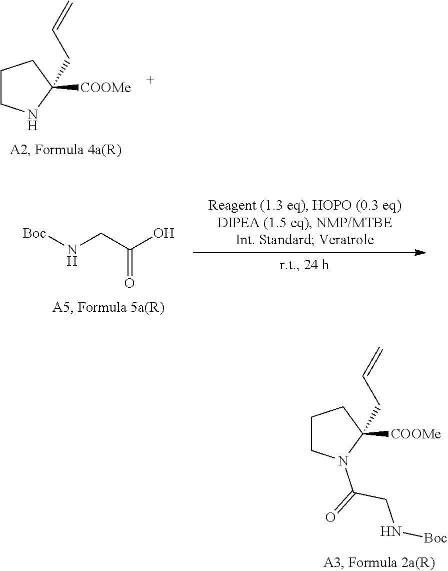 [SMILES: C=CC[C@@]1(C(=O)OC)CCCN1 ; CC(C)(C)OC(=O)NCC(=O)O ; C=CC[C@@]1(C(=O)OC)CCCN1C(=O)CNC(=O)OC(C)(C)C]

A stock solution was made up using 1.000 g of A2, and 1.1155 g of veratrole, and enough MBTE to reach 4.0 mL volume. This was then mixed with NMP to reach 12.0 mL volume. This was used as a stock solution for several screens.

A vial was charged with a specified additive (0.089 mmol, 0.3 eq), (tert-butoxycarbonyl)glycine A5 (56.9 mg, 0.325 mmol, 1.1 eq) and specified amide coupling reagent (0.384 mmol, 1.3 eq) were dissolved in NMP (0.4 mL) with ice bath cooling. Then DIPEA (0.077 mL, 0.443 mmol, 1.5 eq) was added. If the amide coupling reagent was a liquid it was added after NMP, but before DIPEA. This was left to stir for 10 mins with ice bath cooling. To this suspension a stock solution of 0.6 mL of methyl (R)-2-allylpyrrolidine-2-carboxylate A2 (50 mg, 0.295 mmol, 1 eq) and veratrole (0.052 mL, 0.405 mmol, 1.37 eq) in MTBE and NMP was added, also with ice bath cooling. The resulting suspension was left to warm up to ambient temperature (22° C.) and stirred overnight.

After 20 hours, 10 μL of the reaction solution was dissolved in 1 mL MeCN and measured by HPLC.

TABLE 3
Reagents and reaction outcomes

| | | Yield of A3 by quantitative |
|---|---|---|
| Entry | Reagent | HPLC % |
| 1 | EDC•HCl | 79 |
| 2 | DIC | 79 |
| 3 | DCC | 74 |
| 4 | HATU | 81 |
| 5 | HBTU | 80 |
| 6 | TBTU | 80 |
| 7 | CDI | 16 |
| 8 | PivCl | 46 |
| 9 | IBCF | 28 |
| 10 | T₃P | 36 |
| 11 | DppCl | 42 |
| 12 | PyBOP | 80 |

### Example 10: Additive Screen for Amide Coupling

A systematic screen of additives was carried out for the amide coupling.

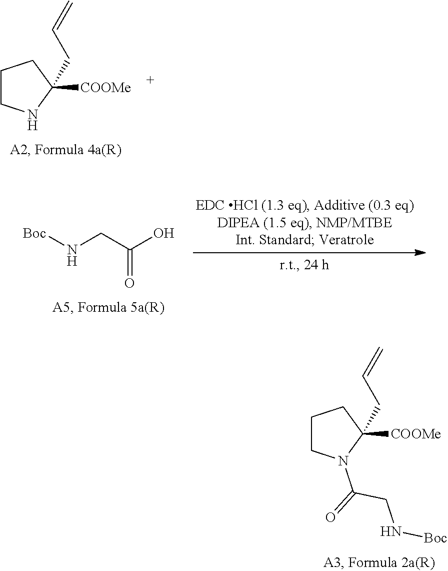 [SMILES: C=CC[C@@]1(C(=O)OC)CCCN1 ; CC(C)(C)OC(=O)NCC(=O)O ; C=CC[C@@]1(C(=O)OC)CCCN1C(=O)CNC(=O)OC(C)(C)C]

A stock solution was made up using 1.000 g of A2, and 1.1155 g of veratrole, and enough MBTE to reach 4.0 mL volume. This was then mixed with NMP to reach 12.0 mL volume. This was used as a stock solution for several screens.

A vial was charged with a specified additive (0.089 mmol, 0.3 eq), (tert-butoxycarbonyl)glycine A5 (56.9 mg, 0.325 mmol, 1.1 eq) and specified amide coupling reagent (0.384 mmol, 1.3 eq) were dissolved in NMP (0.4 mL) with ice bath cooling. Then DIPEA (0.077 mL, 0.443 mmol, 1.5 eq) was added. If the amide coupling reagent was a liquid it was added after NMP, but before DIPEA. This was left to stir for 10 mins with ice bath cooling. To this suspension a stock solution of 0.6 mL of methyl (R)-2-allylpyrrolidine-2-carboxylate A2 (50 mg, 0.295 mmol, 1 eq) and veratrole (0.052 mL, 0.405 mmol, 1.37 eq) in MTBE and NMP was added, also with ice bath cooling. The resulting suspension was left to warm up to ambient temperature (22° C.) and stirred overnight.

After 20 hours, 10 μL of the reaction solution was dissolved in 1 mL MeCN and measured by HPLC.

TABLE 4
Additives and reaction outcomes

| Entry | Additive | Yield of A3 by quantitative HPLC % |
|---|---|---|
| 1 | HOPO | 79 |
| 2 | COMU | 43 |
| 3 | HOBt | 64 |
| 4 | DMAP | 44 |

### Example 11: Coupling of Different PG-Protected Glycines

Alternative PG-protected glycines were explored in the amide coupling reaction.

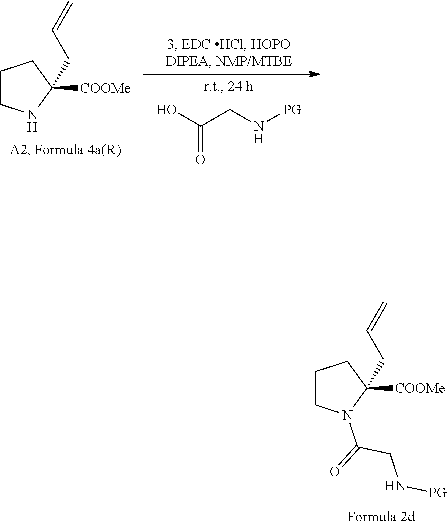

A vial was charged with 2-hydroxypyridine 1-oxide (HOPO) (0.079 g, 0.709 mmol, 0.3 eq), EDC hydrochloride (0.589 g, 3.07 mmol, 1.3 eq) and NMP (1.0 mL) under nitrogen. To this suspension, methyl (R)-2-allylpyrrolidine-2-carboxylate (A2) (0.400 g, 2.364 mmol, 1 eq) in MTBE (0.35 mL) was added, the flask containing A2 was rinsed with MTBE (0.35 mL) and this was added to the reaction mixture at ambient temperature, followed by DIPEA (0.619 mL, 3.55 mmol). The reaction was stirred for 10 min, then placed in an ice-water bath and a solution of PG-protected-glycine (2.60 mmol, 1.1 eq) in NMP (0.7 mL) was added, followed by a rinse with NMP (0.7 mL). The resulting suspension was left to warm up to ambient temperature (22° C.) and stirred overnight.

The reaction was extracted with MTBE (2.5 mL), from citric acid (0.31 g) dissolved in water (2.8 mL). This aqueous layer was extracted further MTBE (3.1 mL×2). The combined organic layers were washed with NaHCO₃ (0.21 g) dissolved in water (4.2 mL). This was dried over Na₂SO₄, filtered and concentrated in vacuo to yield product of Formula 2d for each protecting group (Table 5).

### Fmoc Exception:

A vial was charged with HOBt (0.217 g, 1.418 mmol, 1.2 eq), Fmoc-Gly-OH (0.387 g, 1.300 mmol, 1.1 eq) and DMF (0.5 mL). To this suspension, methyl (R)-2-allylpyrrolidine-2-carboxylate (A2) (0.20 g, 1.182 mmol, 1 eq) in DMF (1 mL) was added, followed by a DMF (1 mL) rinse. This was cooled in an ice bath and then followed by addition of EDC hydrochloride (0.272 g, 1.418 mmol, 1.2 eq). The reaction was allowed to warm to room temperature overnight.

The reaction mixture was poured into ice water (3 mL), and the mixture was extracted with ethyl acetate (3×3 mL). It was dried with Na₂SO₄, filtered and the solvent evaporated to yield a crude oil (1.108 g). This was purified by column chromatography using 12 g silica, with EtO Ac/Heptane (wet loading, with DCM).

Appropriate fractions were pooled and concentrated and the solvent was removed to yield product as a white amorphous solid of methyl (R)-1-((((9H-fluoren-9-yl)methoxy)carbonyl)glycyl)-2-allylpyrrolidine-2-carboxylate.

TABLE 5
PG-protected glycines and reaction outcomes

| | | Isolated yield |
|---|---|---|
| Entry | PG-protected glycine input material | of 2d % |
| 1 | TFA-Gly-OH | 73 |
| 2 | Cbz-Gly-OH | 88 |
| 3 | Trt-Gly-OH | 83 |
| 4 | Fmoc-Gly-OH | 48 |

### Example 12: Coupling with Activated Glycine Esters

The activated glycine esters were also explored in amide coupling reactions.

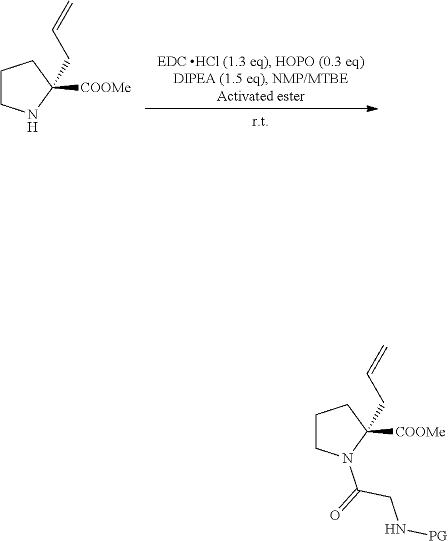

The following activated glycine esters were trialled:

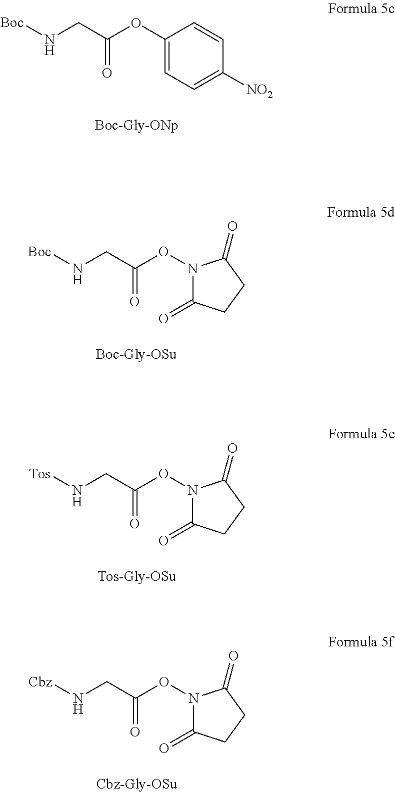

A vial was charged with the activated glycine ester (1.2 eq), EDC hydrochloride (1.3 eq), HOPO (0.3 eq) and cooled with an ice bath. NMP and then DIPEA (1.5 eq) were also added.

Veratrole (only in the cases of 5c and 5d) and methyl (R)-2-allylpyrrolidine-2-carboxylate (A2) (1 eq, scale between 50-250 mg depending on the reaction) were dissolved in NMP. This solution was added to the glycine reaction mixture under ice bath cooling. MTBE was added and the resulting suspension was left to warm up to ambient temperature (22° C.) and stirred overnight.

After 20 hours, 10 μL of the reaction mixture was dissolved in 1 mL MeCN and measured by HPLC.

For the reactions lacking internal standard (5e and 5f):

The reaction was extracted with MTBE (2.5 mL), from citric acid (0.31 g) dissolved in water (2.8 mL). This aqueous layer was extracted further MTBE (3.1 mL×2). The combined organic layers were washed with NaHCO₃ (0.21 g) dissolved in water (4.2 mL). This was dried over Na₂SO₄, filtered and concentrated in vacuo to yield product 2d for each protecting group. If the material was not pure the yield was determined by qNMR.

TABLE 6
Esters and reaction outcomes

| Entry | Activated glycine ester | Yield % |
|---|---|---|
| 1 | Boc-Gly-ONp | 5c: 80 (by qHPLC) |
| 2 | Boc-Gly-OSu | 5d: 50 (by qHPLC) |
| 3 | Tos-Gly-OSu | 5e: 12% yield (by qNMR) |
| 4 | Cbz-Gly-OSu | 5f: 82% (isolated yield) |

### Example 13: Coupling with Acid Chlorides

The possibility to obtain Formula 2d via corresponding acid chlorides was investigated.

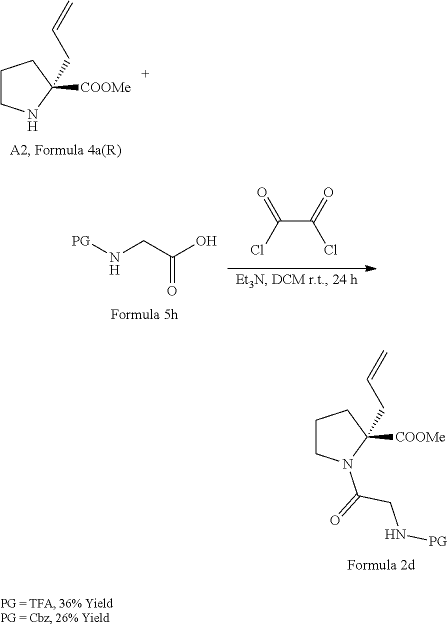

To a solution of N-protected Glycine 5h (1.0 eq) in DCM (0.5 mL), oxalyl chloride (1.2 eq) and a drop of N,N-dimethylformamide were added. The reaction was stirred for 2 h at ambient temperature, gas effervescence stopped.

DCM and an excess of oxalyl chloride were removed in vacuo and the resulting acyl chloride was dissolved in 2 mL DCM and added dropwise to a neat mixture of methyl (R)-2-allylpyrrolidine-2-carboxylate (A2) (100 mg, 1.0 eq) and TEA (1.2 eq) at ambient temperature. The mixture was stirred for 18 h at room temperature. The crude reaction was diluted with water (5 mL) and extracted with DCM (3×5 mL). The organic extracts were combined, dried over Na₂SO₄ and concentrated in vacuo to afford 2d for each protecting group. A qNMR sample was prepared using methyl 3,5-dinitrobenzoate as an internal standard. The TFA-protected derivative of 2d yielded 36% and the Cbz-protected derivative of 2d yielded 26%.

### Example 14: Solvent Screen for Base Initiated Cyclisation

A systematic screen of solvents was carried out for the cyclisation step.

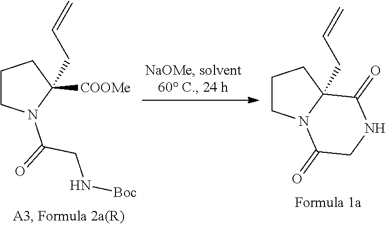 [SMILES: C=CC[C@@]1(C(=O)OC)CCCN1C(=O)CNC(=O)OC(C)(C)C ; C=CC[C@@]12CCCN1C(=O)CNC2=O]

Methyl (R)-2-allyl-1-((tert-butoxycarbonyl)glycyl)pyrrolidine-2-carboxylate A3 (100 mg, 0.306 mmol, 1 eq) was dissolved in 0.5 mL of specified solvent, a stirring bar was added, then the internal standard veratrole (39 μL, 0.306 mmol, 1 eq) and finally sodium methanolate (5.4M, 30 wt. % solution in methanol) (28 μL, 0.153 mmol, 0.5 eq). The vial was sealed, heated to 60° C. and stirred for 24 h. Then, the reaction was cooled to room temperature, 10 μL of the crude mixture was dissolved in 1 mL MeCN and the conversion to 1a, NNZ-2591, measured by HPLC.

TABLE 7
Solvents and reaction outcomes

| | | Yield of 1a by quantitative |
|---|---|---|
| Entry | Solvent | HPLC % |
| 1 | MeOH | 88 |
| 2 | i-PrOH | 73 |
| 3 | NMP | 48 |
| 4 | MeCN | 72 |
| 5 | Toluene | 64 |
| 6 | Pyridine | 73 |
| 7 | MTBE | 33 |
| 8 | DCM | 22 |
| 9 | EtOAc | 98 |
| 10 | Methyl isopropyl ketone (MIPK) | 68 |
| 11 | THF | 70 |
| 12 | DMF | 68 |
| 13 | DMSO | 55 |
| 14 | Dimethyl carbonate (DMC) | 41 |

### Example 15: Base Screen for Base Initiated Cyclisation

A systematic screen of bases was carried out for the cyclisation step.

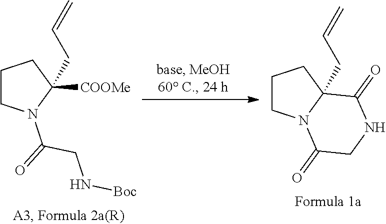 [SMILES: C=CC[C@@]1(C(=O)OC)CCCN1C(=O)CNC(=O)OC(C)(C)C ; C=CC[C@@]12CCCN1C(=O)CNC2=O]

Methyl (R)-2-allyl-1-((tert-butoxycarbonyl)glycyl)pyrrolidine-2-carboxylate A3 (100 mg, 0.306 mmol, 1 eq) was dissolved in MeOH (0.5 mL), a stirring bar was added, then the internal standard veratrole (39 μL, 0.306 mmol, 1 eq) and finally the specified base (1 eq). The vial was sealed, heated to 60° C. and stirred for 24 h. Then, the reaction was cooled to room temperature, 10 μL of the crude mixture was dissolved in 1 mL MeCN and the conversion to 1a, NNZ-2591, measured by HPLC.

TABLE 8
Bases and reaction outcomes

| | | Yield of |
|---|---|---|
| | | 1a by |
| | | quantitative |
| Entry | Base | HPLC |
| 1 | NaOH | 40 |
| 2 | KOH | 44 |
| 3 | NaOₜ—Bu (1M solution in THF) | 56 |
| 4 | NaOEt (21 w/w % solution in EtOH) | 81 |
| 5 | KOₜ—Bu | 68 |
| 6 | LDA (2M solution in THF/heptane/ethylbenzene) | 64 |
| 7 | K₂CO₃ | 66 |
| 8 | LiHMDS (1M in THF) | 77 |
| 9 | NaH (60% dispersion in mineral oil) | 85 |

### Example 16: Cyclisation with Different Protecting Groups (PGs) on Nitrogen

A screen of different protecting groups was carried out under basic or acidic conditions to furnish the cyclisation product Formula 1a.

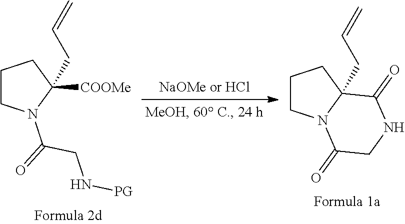

Compound 2d for each protecting group (1 eq) was dissolved in MeOH (0.5 mL), a stirring bar was added, then sodium methanolate (5.4M, 30 wt. % solution in methanol) (0.5 eq). The vial was sealed, heated to 60° C. and stirred (250 rpm) for 24 h. Then, the reaction was cooled to room temperature, 10 μL of the crude mixture was dissolved in 1 mL MeCN and the conversion to 1a, NNZ-2591, measured by HPLC.

TABLE 9
Protecting Group and reaction outcomes

| | | | Yield of |
|---|---|---|---|
| | | | 1a by |
| | Protecting | | quantitative |
| Entry | Group (PG) | Conditions | HPLC % |
| 1 | TFA | 0.5 eq NaOMe (30 w/w % in MeOH) | 88 |
| 2 | Cbz | 0.5 eq NaOMe (30 w/w % in MeOH) | 90 |
| 3 | Fmoc | 0.5 eq NaOMe (30 w/w % in MeOH) | 33 |

1. A process for preparing a bicyclic glycine-proline compound of Formula 1 comprising a base initiated cyclisation reaction of a compound of Formula 2 to form the compound of Formula 1: 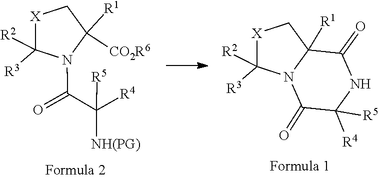
wherein
X is selected from CR⁷R⁸, NR⁷, O, and S;
R⁷ and R⁸ are each independently selected from hydrogen and alkyl;
R¹, R², R³, R⁴, and R⁵, are each independently selected from hydrogen, halogen, alkyl, alkenyl, alkynyl, haloalkyl, —O-haloalkyl, 3-10-membered carbocycle, 3-10-membered heterocycle, —OR⁹, —SR⁹, —NR⁹R¹⁰, —NO₂, —CN, —C(O)R⁹, —C(O)OR⁹, —C(O)NR⁹R¹⁰, and —CNR⁹, or R⁴ and R⁵ taken together provide a 3-10-membered carbocycle; R⁹ and R¹⁰ are each independently selected from hydrogen, —C₁₋₈alkyl, and —C₂₋₈alkenyl;
R⁶ is selected from alkyl, aryl and alkylaryl; and
PG is an amine protecting group.

2. The process of claim 1, wherein X is CR⁷R⁸.

3. The process of claim 1, wherein R⁷ and R⁸ are each hydrogen.

4. The process of claim 1, wherein R², R³, R⁴, and R⁵, are hydrogen.

5. The process of claim 1, wherein R¹ is alkenyl.

6. The process of claim 1, wherein R¹ is —CH₂—CH=CH₂.

7. The process of claim 1, wherein the amine protecting group is a base removable protecting group, preferably wherein the amine protecting group (PG) is selected from trifluoroacetyl (TFA), -Boc (tert-butyloxycarbonyl), -Fmoc (fluorenylmethyloxycarbonyl), and -Cbz (carboxybenzyl).

8. (canceled)

9. The process of claim 1, wherein a base reagent for the base initiated cyclisation reaction has a pKaH of its conjugate acid of at least about 9.

10. The process of claim 1, wherein a base reagent for the base initiated cyclisation reaction is an anionic base, preferably wherein the anionic base is a conjugate base of a Group 1 metal.

11. (canceled)

12. The process of claim 10, wherein the anionic base is selected from a metal alkoxide, metal carbonate, metal hydroxide, metal hydride, metal amine and metal silylamide.

13. The process of claim 1, wherein a base reagent for the base initiated cyclisation reaction is selected from sodium methoxide (NaOMe), sodium ethoxide (NaOEt), sodium isopropoxide, sodium tert-butoxide (NaOtBu), sodium bis(trimethylsilyl)amide (NaHMDS), lithium bis(trimethylsilyl)amide (LiHMDS), sodium hydride (NaH), sodium hydroxide (NaOH), potassium hydroxide (KOH), potassium carbonate, sodium carbonate, lithium diisopropylamide (LDA), potassium methoxide (KOMe), potassium ethoxide (KOEt), potassium isopropoxide, potassium tert-butoxide (KOtBu), and any combinations thereof.

14. (canceled)

15. The process of claim 1, wherein a base reagent for the base initiated cyclisation reaction is sodium methoxide (NaOMe) provided in molar equivalents of between about 0.01 and 5, between about 0.05 and 4, or between about 0.1 and 2, or between about 0.5 to 1.5, relative to the compound of Formula 2.

16. (canceled)

17. The process of claim 1, wherein the cyclisation reaction is performed in the presence of a polar protic solvent, an aprotic solvent, or a non-polar solvent, preferably wherein the polar protic solvent is an alcohol selected from methanol, ethanol and isopropyl alcohol, or any combinations thereof, and wherein the aprotic solvent or non-polar solvent is selected from hydrocarbons, halogenated hydrocarbons, aromatic hydrocarbons, ketones, nitriles, esters, carbonate esters, ethers, sulfoxides, sulfones, amides, nitroalkanes, pyrrolidines, or any combinations thereof, preferably wherein the aprotic solvent or non-polar solvent is selected from tetrahydrofuran (THF), methyltetrahydrofuran (MeTHF), acetonitrile (MeCN), N-methylpyrrolidone (NMP), pyridine, toluene, hexanes, n-heptane, ethyl acetate (EtOAc), methylisopropyl ketone (MIPK), N,N-dimethylformamide (DMF), dimethylsulfoxide (DMSO), or any combinations thereof.

18.-23. (canceled)

24. The process of claim 1, wherein the bicyclic glycine-proline compound of Formula 1 is a bicyclic glycine-proline compound of Formula 1a: 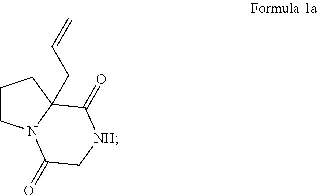
preferably wherein the bicyclic glycine-proline compound of Formula 1 is a bicyclic glycine-proline compound of Formula 1a(R) or Formula 1a(S):

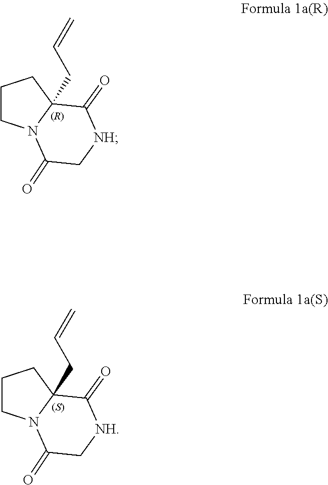

25.-26. (canceled)

27. The process of claim 1, wherein the process for preparing an amide compound of Formula 2: 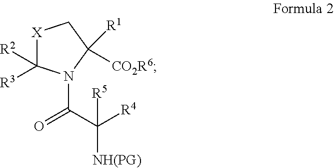
comprises reacting a compound of Formula 4 or salt thereof: 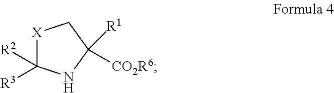
with a compound of Formula 5: 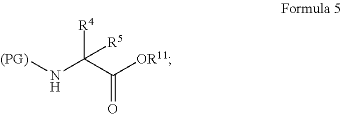
under amide coupling conditions, wherein
X is selected from CR⁷R⁸, NR⁷, O, and S;
R⁷ and R⁸ are each independently selected from hydrogen and alkyl;
R¹, R², R³, R⁴, and R⁵, are each independently selected from hydrogen, halogen, alkyl, alkenyl, alkynyl, haloalkyl, —O-haloalkyl, 3-10-membered carbocycle, 3-10-membered heterocycle, —OR⁹, —SR⁹, —NR⁹R¹⁰, —NO₂, —CN, —C(O)R⁹, —C(O)OR⁹, —C(O)NR⁹R¹⁰, and —CNR⁹, or R⁴ and R⁵ taken together is a 3-10-membered carbocycle; R⁹ and R¹⁰ are each independently selected from hydrogen, —C₁₋₈alkyl, and —C₂₋₈alkenyl;
R⁶ is selected from alkyl, aryl and alkylaryl;
PG is an amine protecting group;
R¹¹ is selected from H and AG; and
AG is an activating group.

28. The process of claim 27, wherein the process is performed in a polar aprotic solvent or non-polar solvent, preferably wherein the polar aprotic solvent or non-polar solvent is selected from N-methyl-2-pyrrolidone (NMP), methyl-tert-butyl ether (MTBE), pyridine, dimethylformamide (DMF), ethyl acetate (EtOAc), toluene, methyltetrahydrofuran (MeTHF), dimethylsulfoxide (DMSO), dioxane, acetone or any combination thereof.

29. (canceled)

30. The process of claim 27, wherein the amide coupling conditions comprise an amide coupling reagent, wherein the amide coupling reagent is selected from 1-ethyl-3-(3-dimethylaminopropyl)carbodiimide (EDC), dicyclohexylcarbodiimide (DCC), diisopropylcarbodiimide (DIC), propyl phosphonic anhydride (T₃P), Hexafluorophosphate Azabenzotriazole Tetramethyl Uronium (HATU), benzotriazole-N,N,N′,N′-tetramethylurea hexafluorophosphate (HBTU), 2-(1H-benz′triazo L-1-yl)-1,′,3,3-Tetramethylurea tetrafluoroborate (TBTU), N,N′-carbonyldiimidazole (CDI), pivaloyl chloride (PivCI), Isobutyl chloroformate (IBCF), cyanuric chloride (TCT), diphenylphosphinic acid chloride (DppCl), 1H-benzotriazol-1-yloxytripyrrolidinyl hexafluorophosphate (PyBOP) or salts thereof, or any combinations thereof.

31.-35. (canceled)

36. The process of claim 27, wherein the process for preparing a compound of Formula 4 or salt thereof: 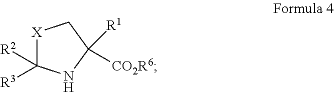
comprises reacting a compound of Formula 6 or salt thereof: 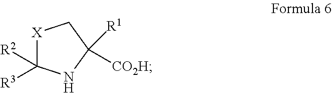
under acid-catalysed esterification conditions, wherein
X is selected from CR⁷R⁸, NR⁷, O, and S;
R⁷ and R⁸ are each independently selected from hydrogen and alkyl;
R¹, R², and R³ are each independently selected from hydrogen, halogen, alkyl, alkenyl, alkynyl, haloalkyl, —O-haloalkyl, 3-10-membered carbocycle, 3-10-membered heterocycle, —OR⁹, —SR⁹, —NR⁹R¹⁰, —NO₂, —CN, —C(O)R⁹, —C(O)OR⁹, —C(O)NR⁹R¹⁰, and —CNR⁹; R⁹ and R¹⁰ are each independently selected from hydrogen, —C₁₋₈alkyl, and —C₂₋₈alkenyl; and
R⁶ is selected from alkyl, aryl and alkylaryl.

37. The process of claim 36, wherein the acid-catalysed esterification conditions include the presence of a reagent to catalyse the esterification, preferably wherein the reagent is selected from methanesulfonic acid, hydrogen chloride, sulfuric acid, thionyl chloride, acetyl chloride, trimethylsilyl chloride, and oxalyl chloride.

38.-42. (canceled)

43. The process of claim 36, wherein the salt of Formula 4 is the hydrochloride salt.
